(19) United States
(12) Patent Application Publication
(10) Pub. No.: US 2025/0349006 A1
(43) Pub. Date: Nov. 13, 2025

(54) METHOD AND SYSTEM FOR DIAGNOSING DISEASE USING MEDICAL IMAGING DATA

(71) Applicant: Shahid A. Abbasi, Riverwoods, IL (US)

(72) Inventors: Shahid A. Abbasi, Riverwoods, IL (US); Mohammad Kamran Khan, Chalyar (PK); Shabbir Ahmad Khan, Stockton, CA (US); Mahd Bin Naeem, Islamabad (PK)

(73) Assignee: Xylexa Inc, Riverwoods, IL (US)

(21) Appl. No.: 19/277,326

(22) Filed: Jul. 22, 2025

(51) Int. Cl. G06T 7/00; A61B 6/03; A61B 6/50; G06F 21/62; G06T 7/11; G06V 10/25; G06V 10/26; G06V 10/40; G06V 10/72; G06V 10/764; G06V 10/774; G06V 10/82; G06V 20/50; G06V 20/70; G16H 15/00; G16H 30/20; G16H 50/20

(52) U.S. Cl. CPC G06T 7/0012; A61B 6/032; A61B 6/504; G06F 21/6254; G06T 7/11; G06V 10/25; G06V 10/267; G06V 10/40; G06V 10/72; G06V 10/764; G06V 10/774; G06V 10/82; G06V 20/50; G06V 20/70; G16H 15/00; G16H 30/20; G16H 50/20; G06T 2207/10081; G06T 2207/20081; G06T 2207/20084; G06T 2207/30104; G06V 2201/03

(57) ABSTRACT

Methods and systems are disclosed for using medical imaging data to diagnose peripheral arterial disease. In a method, a plurality of artificial intelligence based neural network models are trained on medical imaging data of a large population of anonymous patients after labeling and structuring the data for training and testing purposes. Medical imaging data of a known patient is then processed by the plurality of pre-trained artificial intelligence based neural network models to diagnose peripheral arterial disease. A rule-based algorithm integrates the predictions made by the pre-trained neural network models. An inference engine analyzes the integrated predictions data for the known patient, detects any anomalies in the pixel intensities present in each medical image, and performs volumetric calculations. A report generation engine generates medical reports for the known patient. A visualization tool enables a clinician to display and view the results of the diagnoses superimposed on medical images.

### BACKGROUND OF THE INVENTION

The subject matter disclosed herein relates to systems, methods, and devices for medical image analysis, diagnosis, risk analysis, decision-making, and/or disease tracking.

Traditional methods use medical imaging data to assist in diagnosing disease. Multiple images are captured during the scanning process as the machine rotates around a patient. Each medical imaging vendor provides a software for receiving medical images over a network and saving additional parameters including patient's name, age, height, scan protocol (e.g., head, chest, abdomen, pelvis), slice thickness, contrast type, and other specifications.

A technician processes and reconstructs these images into detailed 3D representations of the arteries using an image manipulation software while changing some parameters (e.g., changing slice thickness from 1 mm to 0.5 mm). Images are uploaded to a Picture Archiving and Communication System (PACS)—a medical imaging technology for securely storing and digitally transmitting electronic images along with clinically pertinent data. These images can be viewed using any publicly available viewer (e.g., Weasis, or Radiant) for displaying Digital Imaging and Communications in Medicine (DICOM) images.

A human radiologist carefully examines these images and meticulously analyzes all blood vessels for any abnormalities (including blockages, narrowing, or aneurysms) pointing to one or more underlying vascular issues. Radiologist prepares a comprehensive Electronic Medical Record (EMR) report based on observed abnormalities and provides additional measurements like the degree of stenosis (narrowing) of the arteries. A physician or a surgeon reviews EMR report of a patient to fully understand vascular health status.

However, there are problems with the existing methods for analyzing medical images of a patient, diagnosing, and reporting disease. Reviewing and analyzing large number of images is time-consuming. Additionally, DICOM viewers/tools for detecting blockages or artery narrowing may not always provide optimal accuracy. These limitations highlight areas where further improvements in technology and/or processes could enhance the overall accuracy, efficacy and reliability of vascular imaging. Accordingly, there is a need in the art for improved systems, methods, and devices.

### SUMMARY OF THE INVENTION

The present invention relates to computer-based diagnosis of vascular disease, and more particularly to computer-based artificial intelligence-based diagnosis of peripheral artery disease.

Embodiments of the present invention may include a method for receiving medical images as DICOM files of a large population of anonymized patients (to conform with HIPAA rules) from a medical image scanner.

Embodiments may also include a method for extracting arterial phase views of anonymized patients from the DICOM database, saving axial view images, and converting axial view images for said anonymized patients into a 2D PNG image format suitable for further processing. Converted PNG image data may be structured and used to train a neural network recognition model for predicting and recognizing different regions of arterial classes in a human Peripheral Arterial System.

Embodiments may include one or more methods for training a plurality of neural network models including classification, segmentation, and artery labeling models. For each region of the peripheral arterial system, a separate neural network classification model is trained to learn how to categorize each one of the arterial classes present in the images dataset for said region. Similarly, a separate neural network segmentation model is trained to learn how to segment images and create image masks for all images present in the images dataset for said region. Similarly, a separate neural network artery labeling model for the arterial classes and arteries in them is trained to learn how to label detected objects and predict bounding boxes for these objects in all images and/or in their image masks that may be present in the images dataset for said region.

Embodiments of the present invention may also include a method for receiving DICOM files of a known patient, extracting a plurality of axial view images from the arterial phase of the scanning process, and converting these axial view images into 2D PNG image format suitable for further processing for the known patient. Embodiments of the present invention may also include running the neural network recognition model on the converted axial view images of the known patient to predict one or more regions of the peripheral arterial system of said known patient.

Embodiments may include methods for processing images of said known patient for each region of the peripheral arterial system separately in an iterative manner. For processing images of the known patient for each region of the peripheral arterial system, a separate classification models is used for said region to categorize each one of the arterial classes present in the images dataset in said region for the known patient. Similarly, a separate neural network segmentation model is used to segment images and create image masks for all images present in the images dataset for said region for said known patient.

Embodiments may include a noise filtering process for removing inconsistencies or noise from all images and image masks of predicted arterial classes of a region of the peripheral arterial system prior to generating a resultant arterial image by applying a Bitwise AND Operation on each image and its corresponding image mask for said known patient.

Embodiments may include a process for labeling detected objects in each resultant arterial image and drawing boundary boxes around the detected objects in each resultant arterial image of said known patient.

Embodiments may include an Anekanta Algorithm method for integrating partial predictions made by a plurality of neural network models prior to feeding the results to an inference engine for diagnosing disease in the peripheral arterial system of said known patient, and to a report generation engine for medical reporting purposes.

Embodiments may also include one or more methods for visualizing predicted results after superimposing them on top of said known patient's images in a plurality of views suitable for display on an industry-standard DICOM viewer.

In some embodiments of the present invention, medical images of a known patient are captured and processed at different points in time to diagnose and report the progress or remission of the disease for the known patient.

These and other advantages of the invention will be apparent to those of ordinary skill in the art by reference to the following detailed description and the accompanying drawings.

### BRIEF DESCRIPTION OF THE DIAGRAMS

Claimed subject matter is particularly pointed out and distinctly claimed in the concluding portion of the specification. However, both as to organization and/or method of operation, together with objects, features, and/or advantages thereof, may best be understood by reference to the following detailed description when read with the accompanying drawings in which:

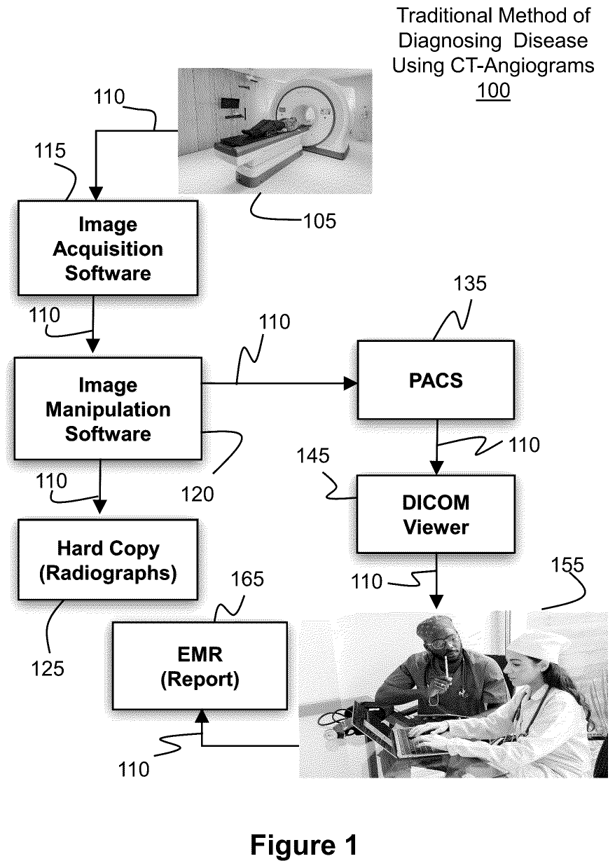

FIG. 1 illustrates a traditional system for diagnosing disease using CT-Angiograms.

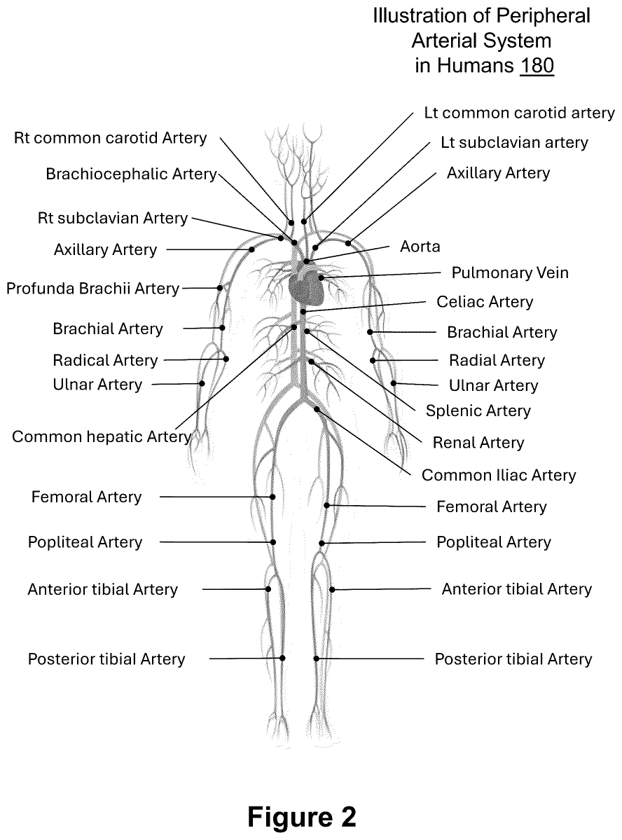

FIG. 2 illustrates Peripheral Arterial System in a human body.

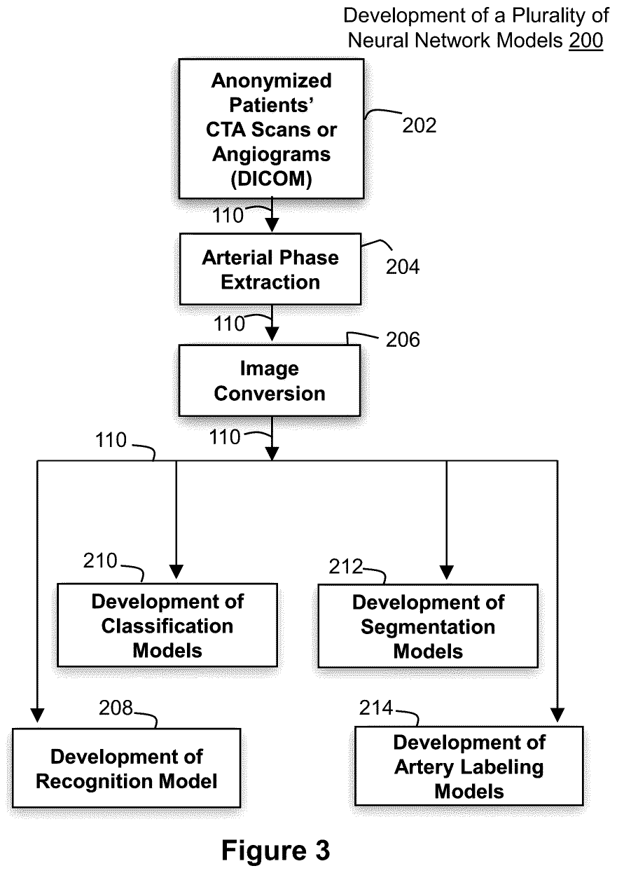

FIG. 3 illustrates a high-level block diagram of a preferred embodiment of the invention for developing a plurality of neural network models.

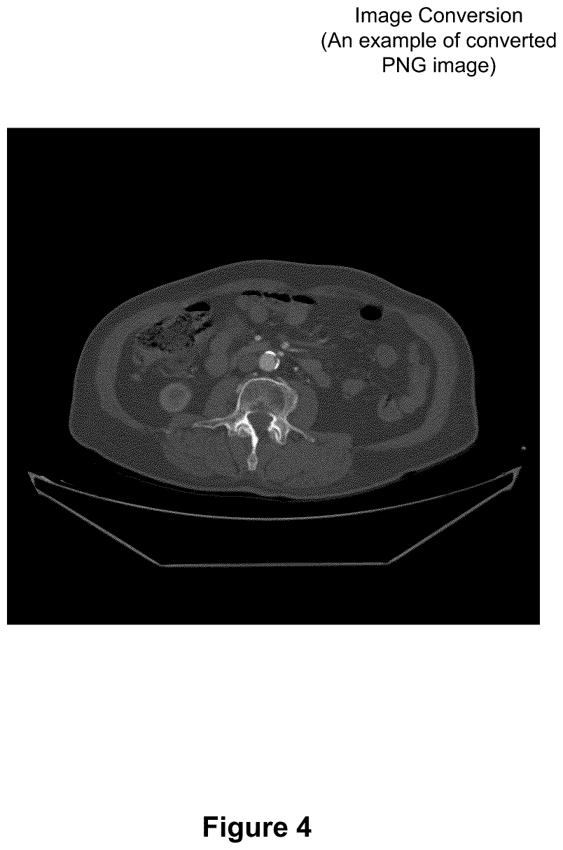

FIG. 4 illustrates the result of an image conversion method to convert a DICOM image into a PNG image.

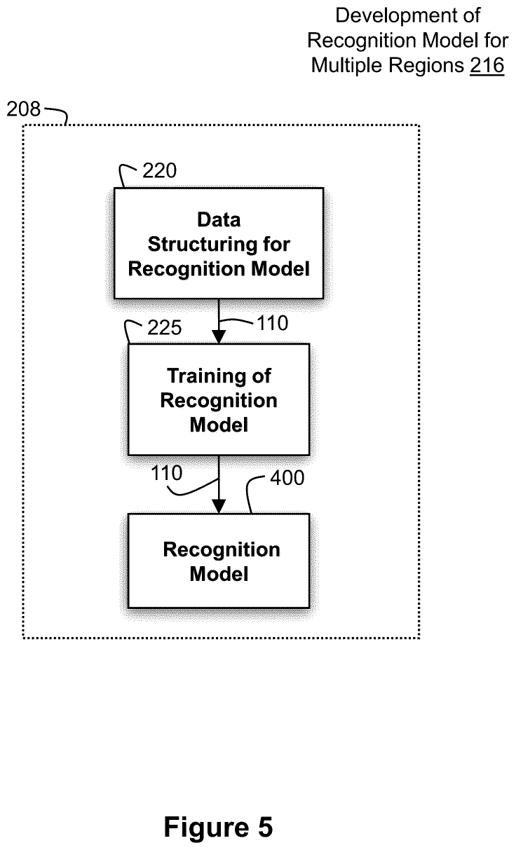

FIG. 5 illustrates a high-level block diagram of developing a neural network Recognition Model.

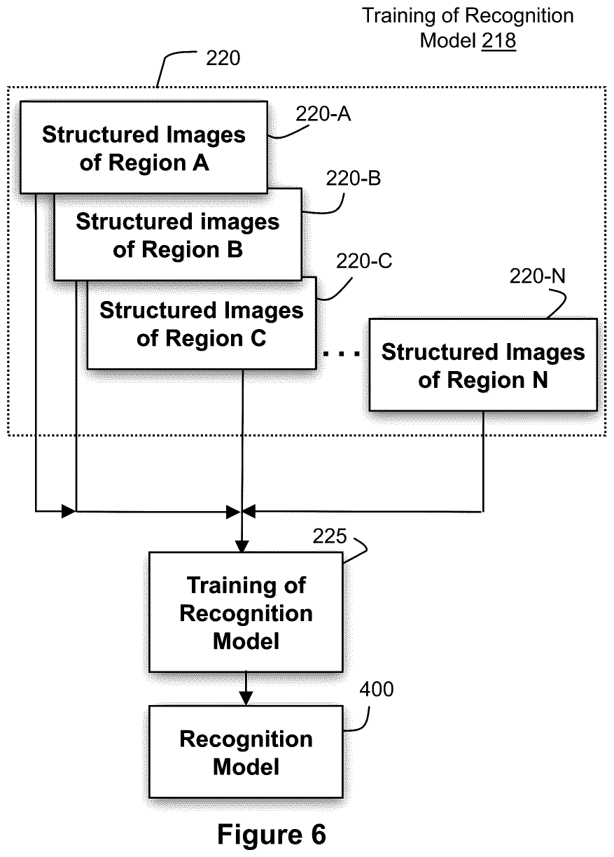

FIG. 6 is a high-level block diagram illustrating the training of a residual neural network Recognition Model by using a plurality of images datasets where each dataset corresponds to a specific arterial region.

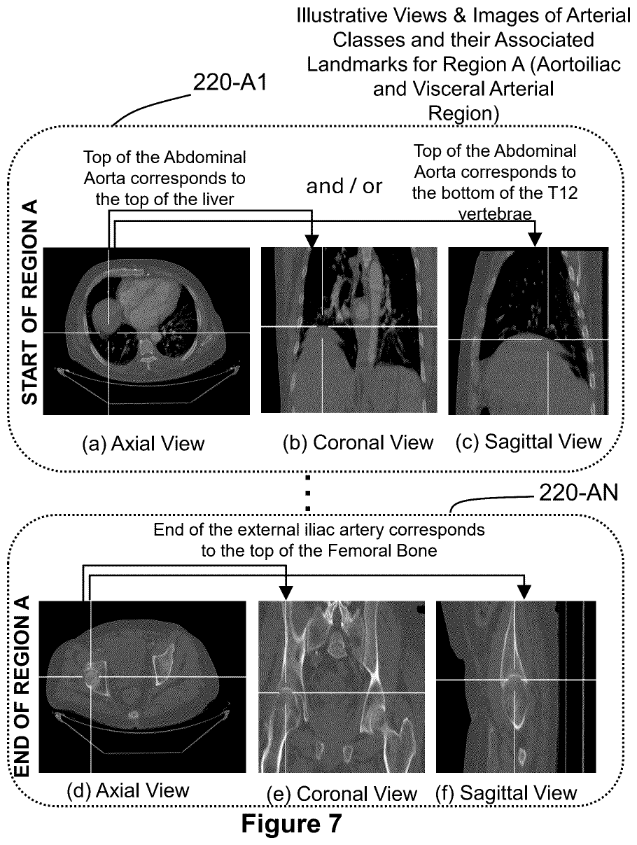

FIG. 7 illustrates the use of all three types of views (i.e., axial, coronal, and sagittal) as an example of using landmarks to mark the starting image and ending image of an arterial region.

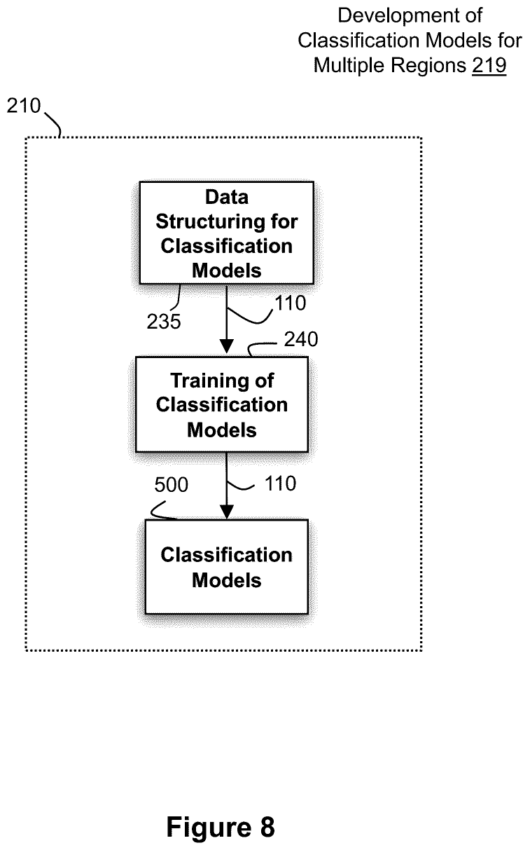

FIG. 8 illustrates a high-level block diagram of developing a plurality of residual neural network Classification Models.

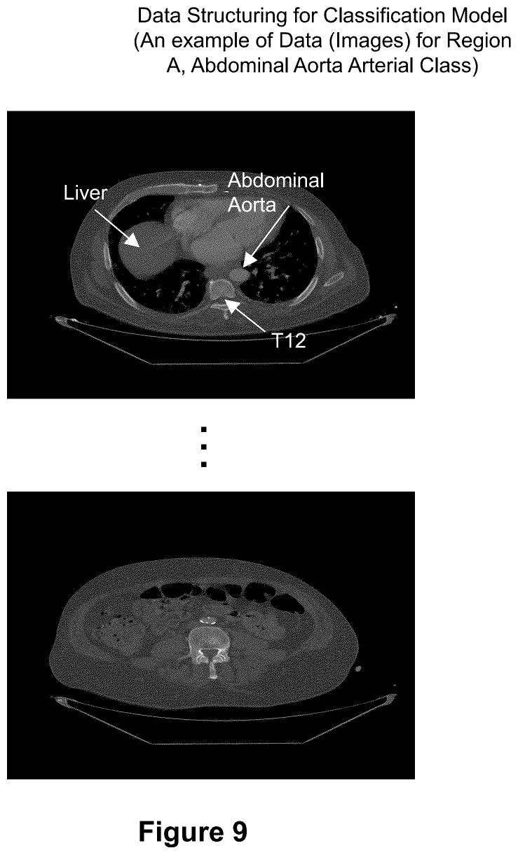

FIG. 9 illustrates an example of data structuring of an arterial class (abdominal aorta) that may be stored in a separate folder.

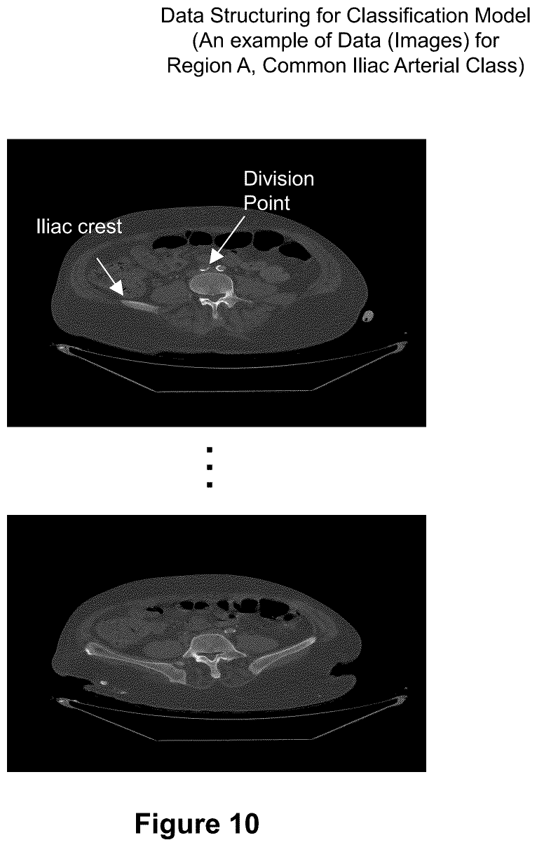

FIG. 10 illustrates an example of data structuring of an arterial class (common iliac arteries) that may be stored in a separate folder.

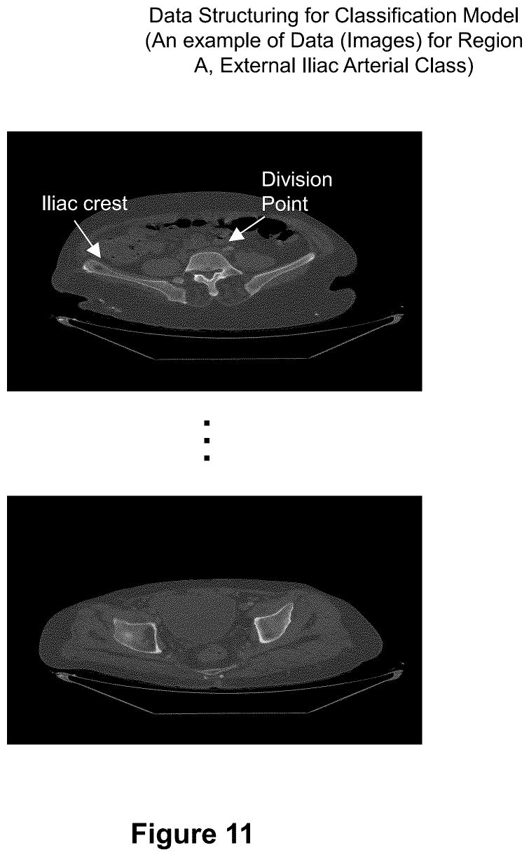

FIG. 11 illustrates an example of data structuring of an arterial class (external iliac arteries) that may be stored in a separate folder.

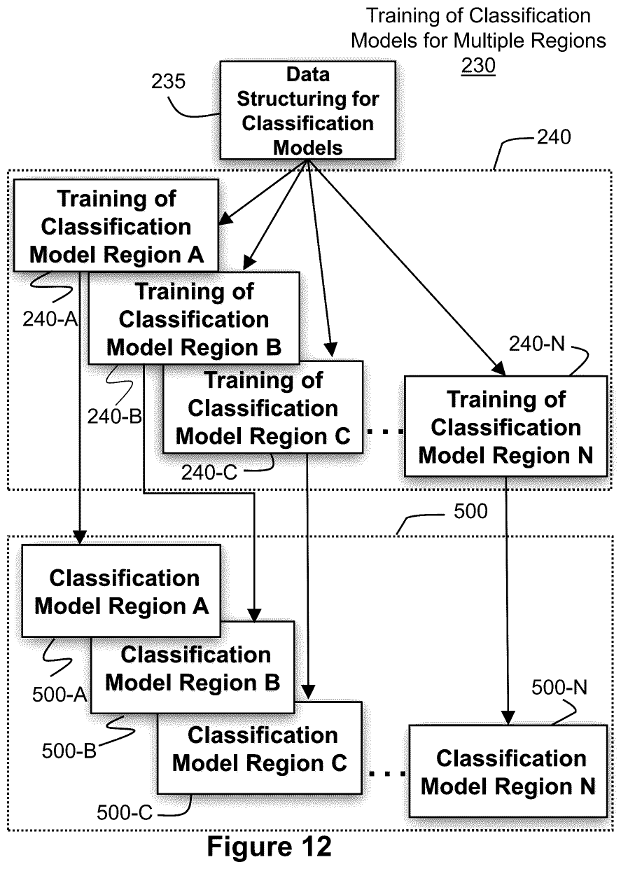

FIG. 12 is a high-level block diagram illustrating the training of a plurality of residual neural network Classification Models where each model corresponds to a specific arterial region.

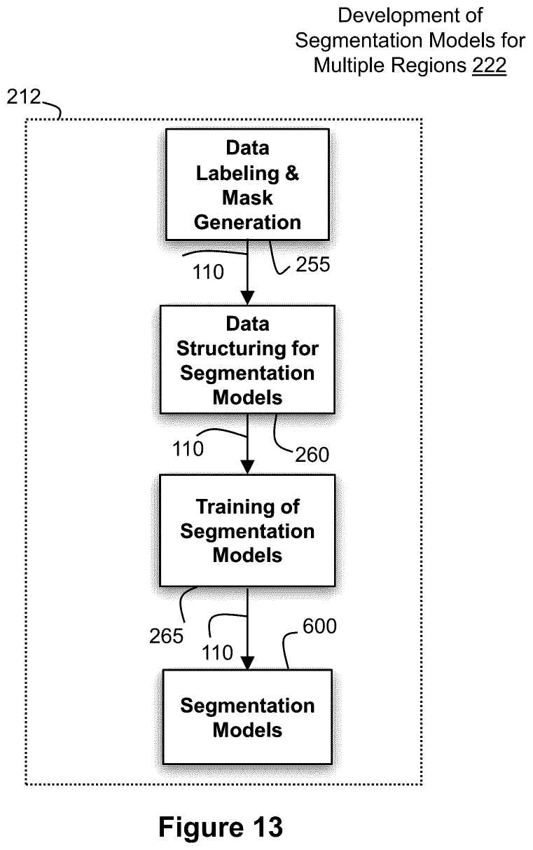

FIG. 13 illustrates a high-level block diagram of developing a plurality of residual neural network Segmentation Models.

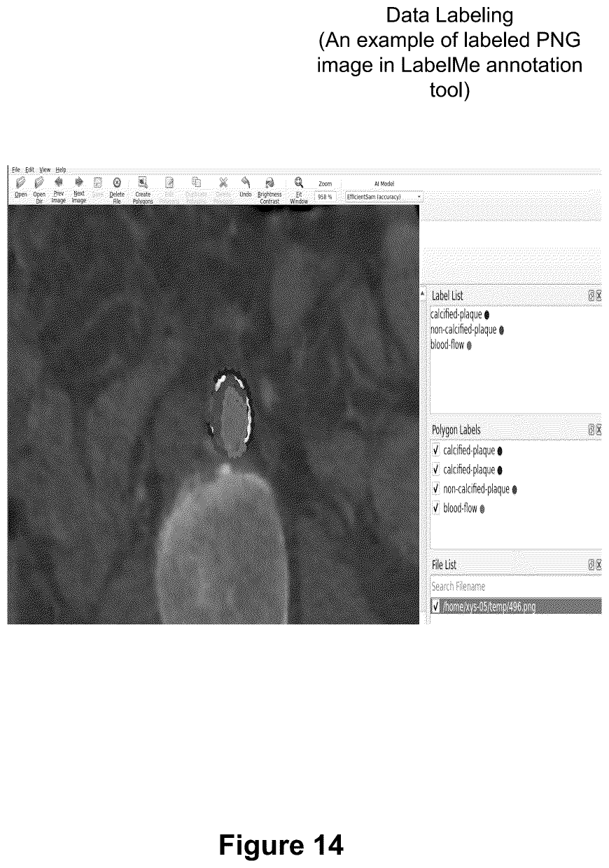

FIG. 14 illustrates an example of data labeling and mask generation of an unlabeled image where the boundary of an arterial class and/or artery (member of an arterial class) and a plurality of features within it have been labeled by zooming into it.

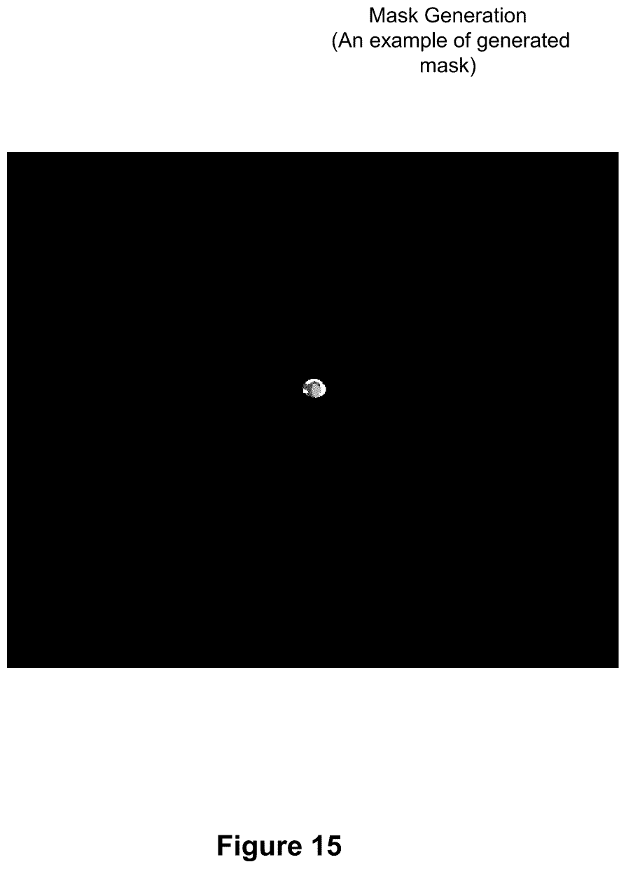

FIG. 15 illustrates an example of a mask generated from an image.

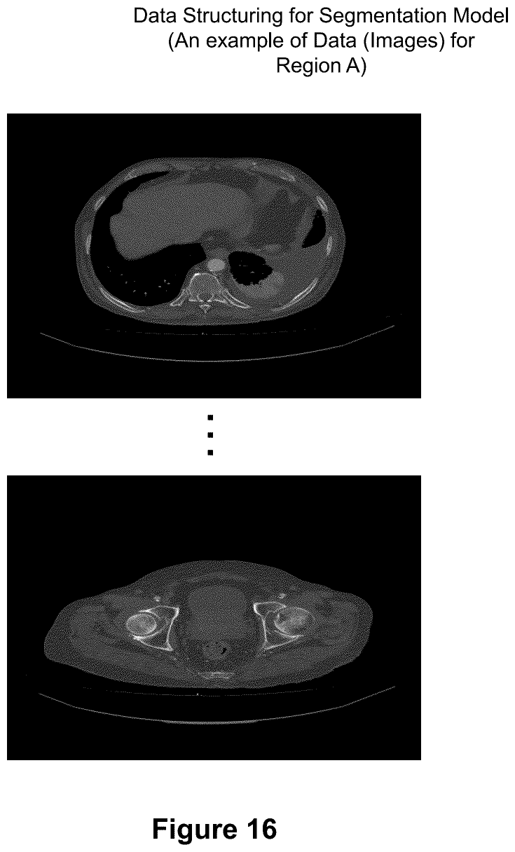

FIG. 16 illustrates an example of input images for a region of the peripheral arterial system.

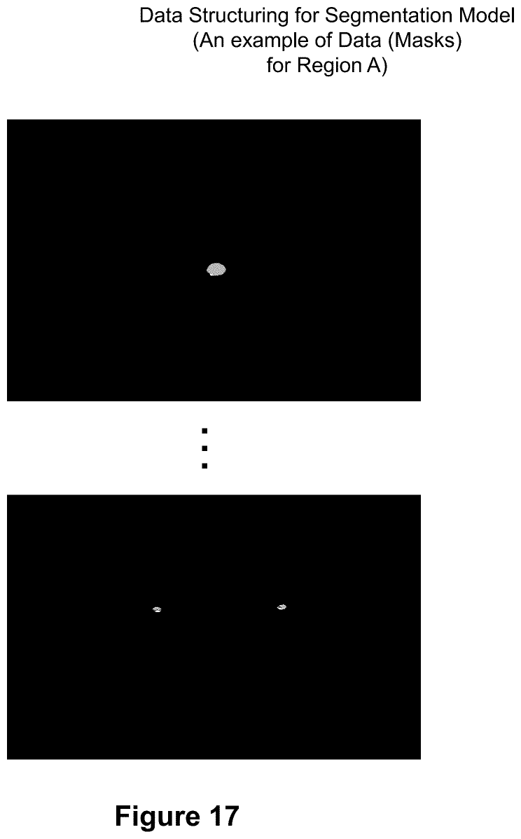

FIG. 17 illustrates an example of masks generated for a region of the peripheral arterial system.

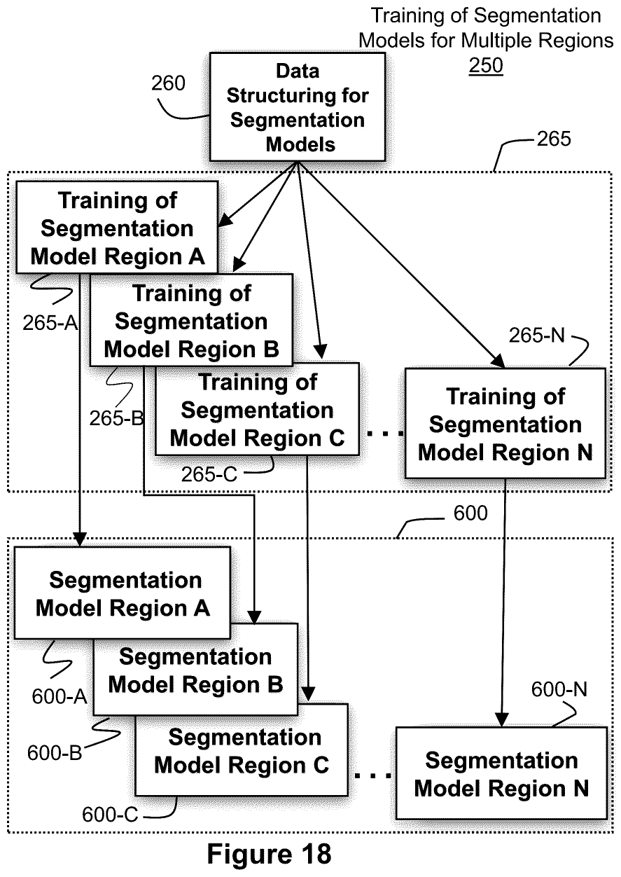

FIG. 18 is a high-level block diagram illustrating the training of a plurality of residual neural network Segmentation Models where each model corresponds to a specific arterial region.

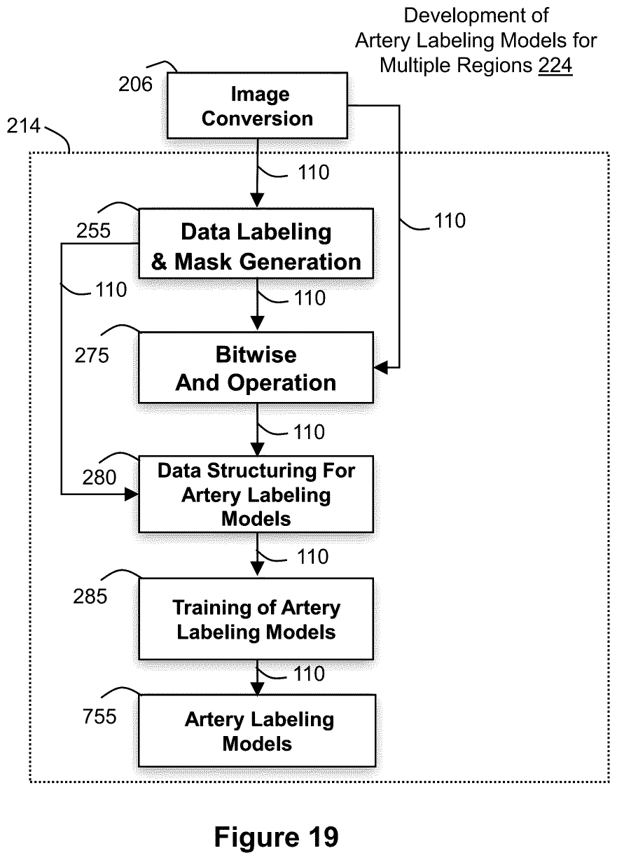

FIG. 19 illustrates a high-level block diagram of developing a plurality of residual neural network Artery Labeling Models where each model corresponds to a specific arterial region.

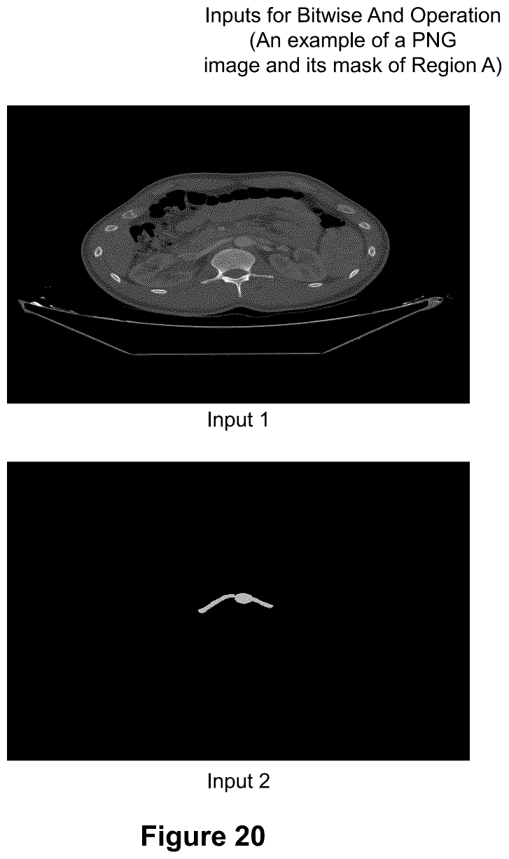

FIG. 20 illustrates an example of an input image and its mask for feeding to a Bitwise AND Operation method.

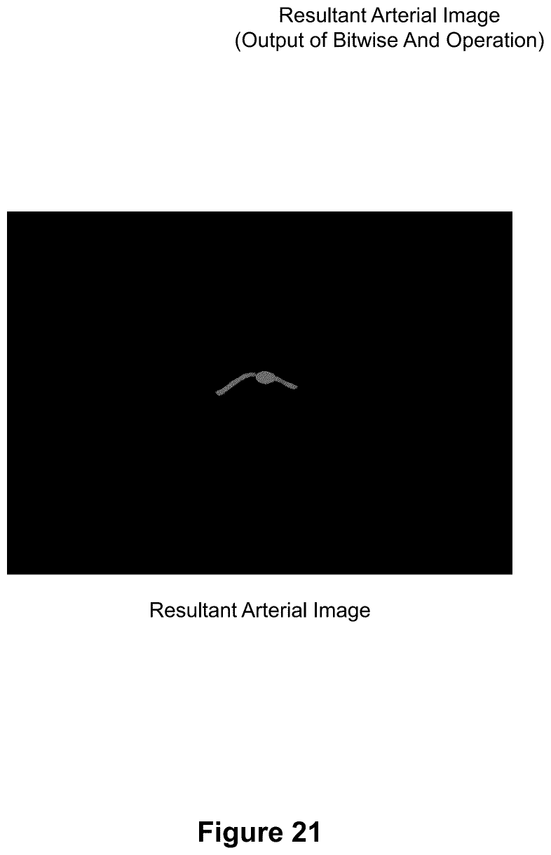

FIG. 21 illustrates an Arterial Arterial Image generated as a result of the Bitwise AND Operation method.

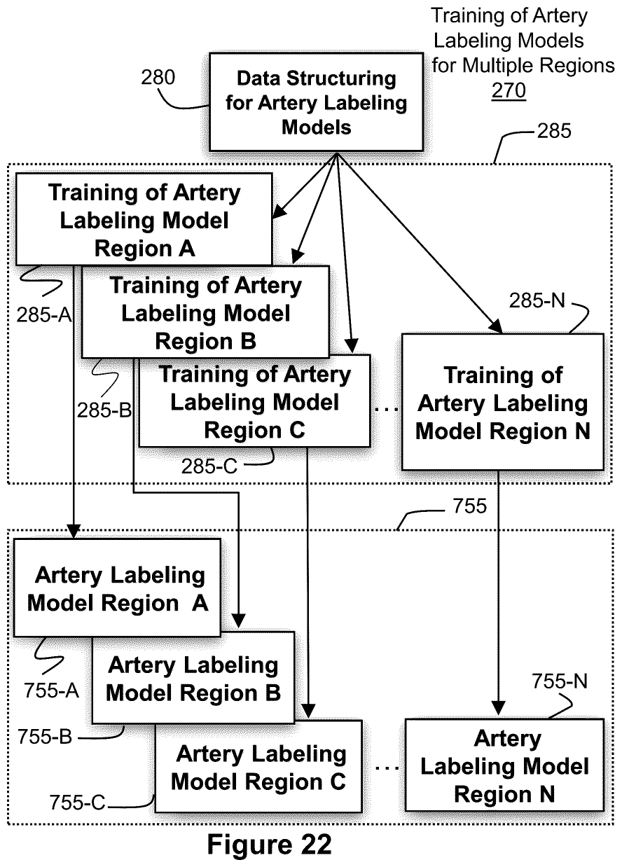

FIG. 22 is a high-level block diagram illustrating the training of a plurality of residual neural network Artery Labeling Models where each model corresponds to a specific arterial region.

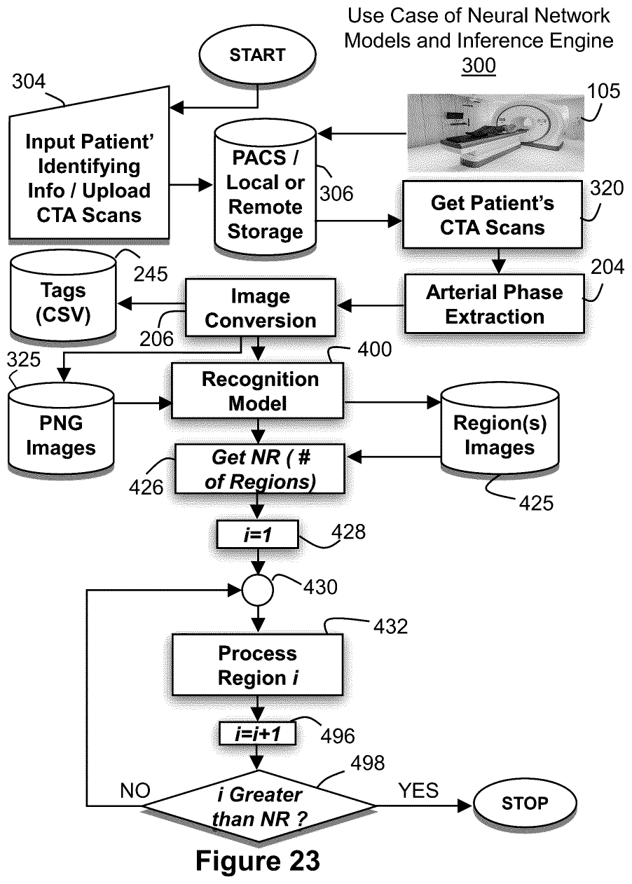

FIG. 23 is a high-level block diagram illustrating a use case of pre-trained residual neural network models for diagnosing peripheral arterial disease for a known patient.

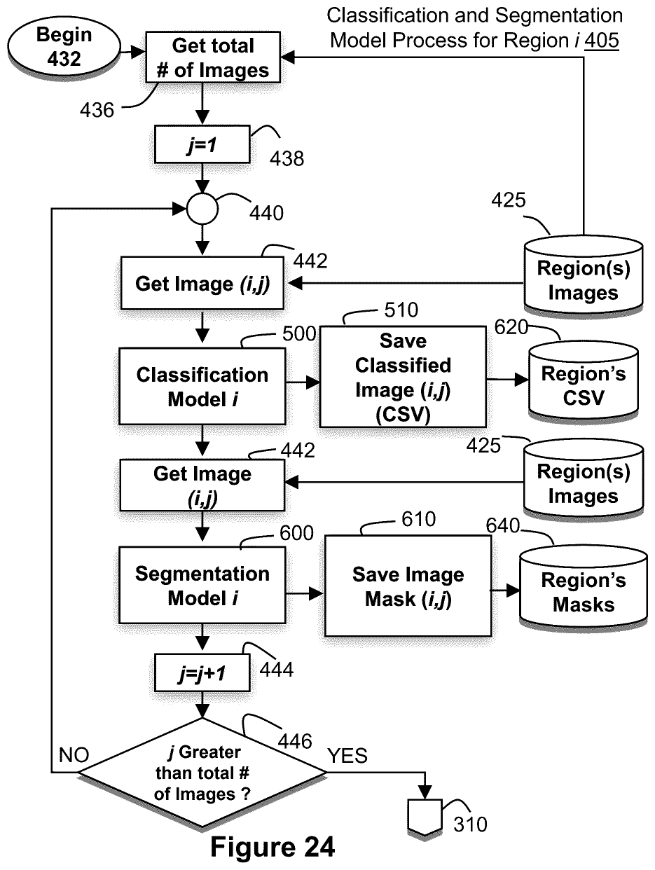

FIG. 24 is a high-level flowchart illustrating the use of pre-trained residual neural network classification model, and a segmentation model, for a region of the peripheral arterial system of a known patient.

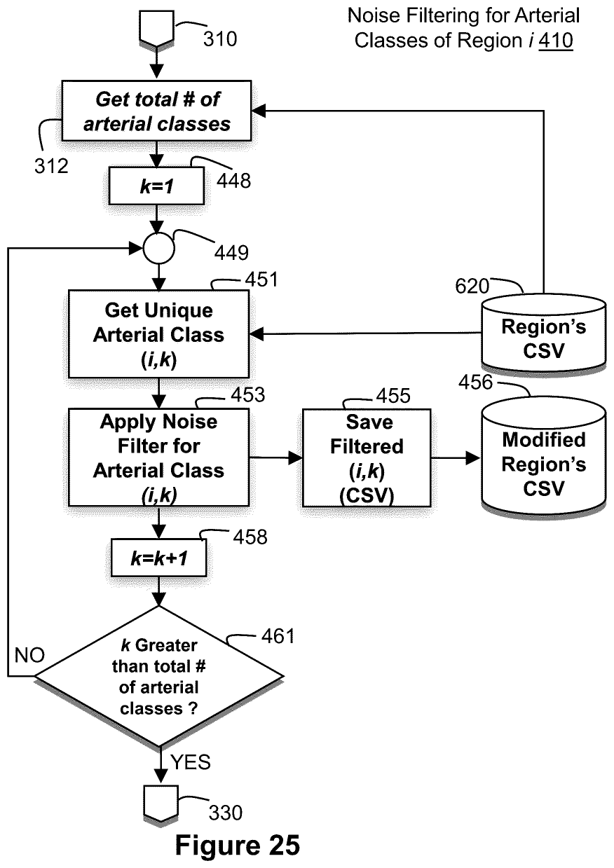

FIG. 25 is a high-level flowchart illustrating a process for removing inconsistencies or noise from the results predicted by the pre-trained residual neural network classification model for a region of the peripheral arterial system of a known patient.

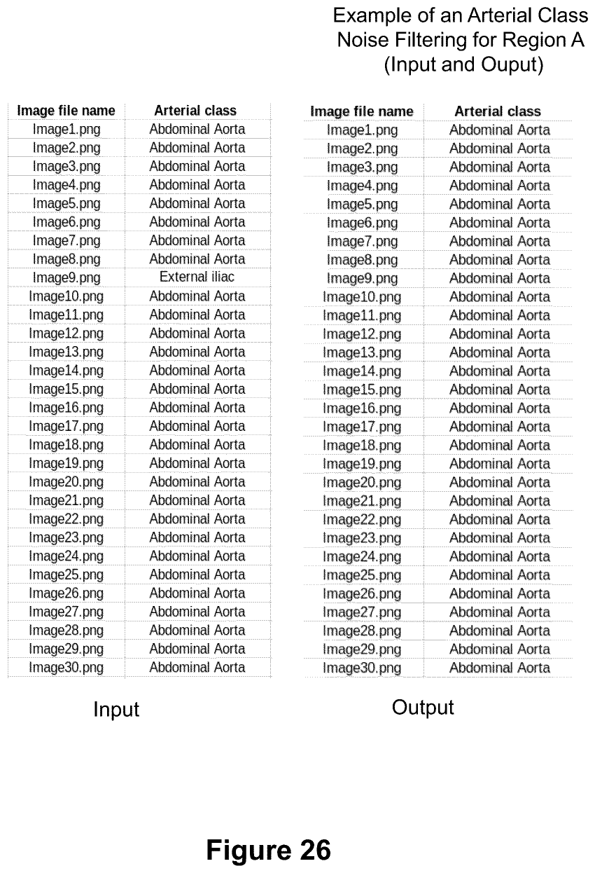

FIG. 26 illustrates an example of an inconsistency in the classified results (shown as an input to the noise filtering process) and the classified results after removing the noise or inconsistency (shown as an output).

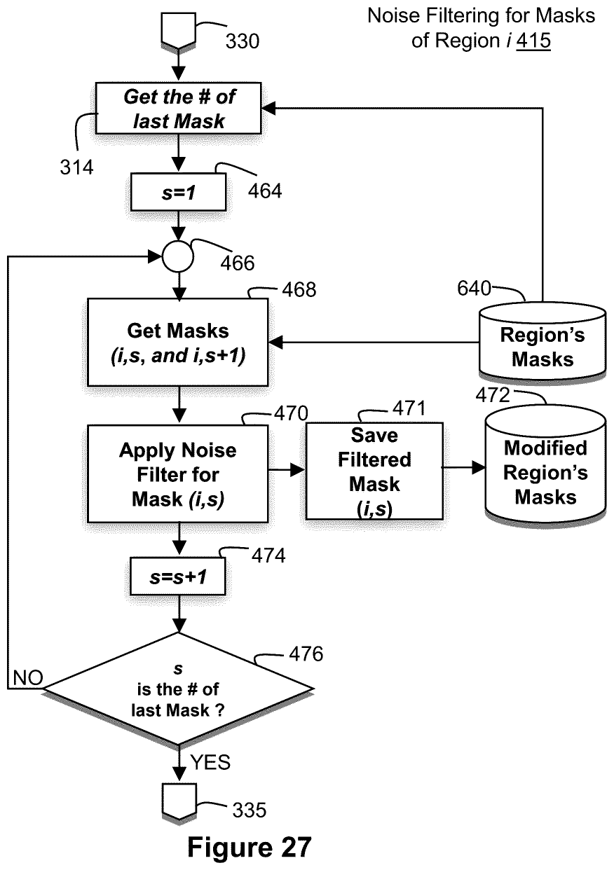

FIG. 27 is a high-level flowchart illustrating a process for removing inconsistencies or noise from the results predicted by the pre-trained residual neural network segmentation model for a region of the peripheral arterial system.

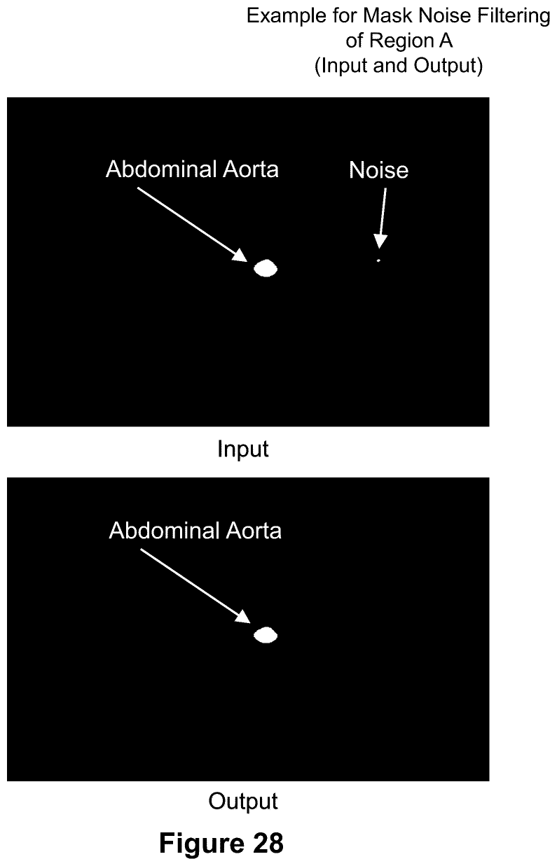

FIG. 28 illustrates an example of an inconsistency in an image mask (shown as an input to the noise filtering process) and the resulting image mask after removing the noise or inconsistency (shown as an output).

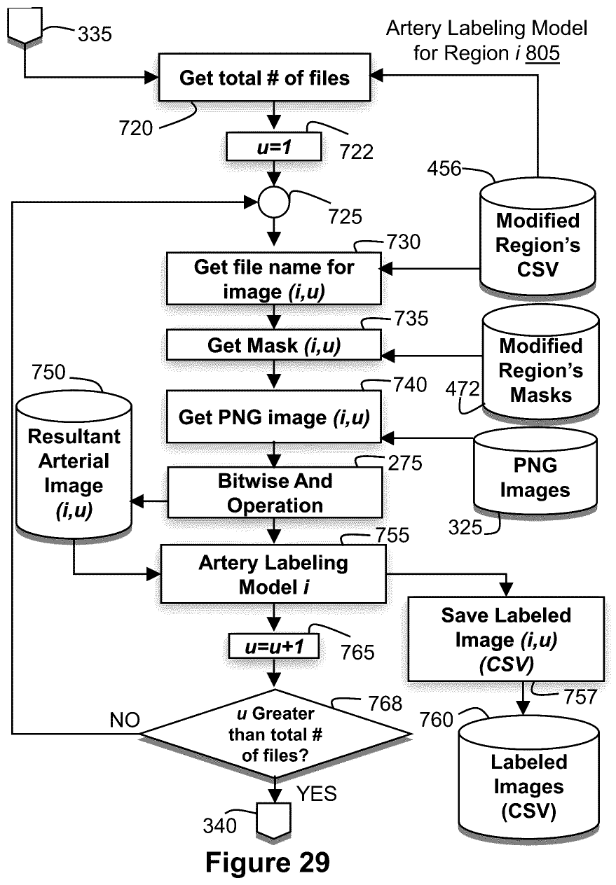

FIG. 29 is a high-level flowchart illustrating a process for identifying and labeling arteries by utilizing methods including a pre-trained residual neural network artery labeling model for a region of the peripheral arterial system.

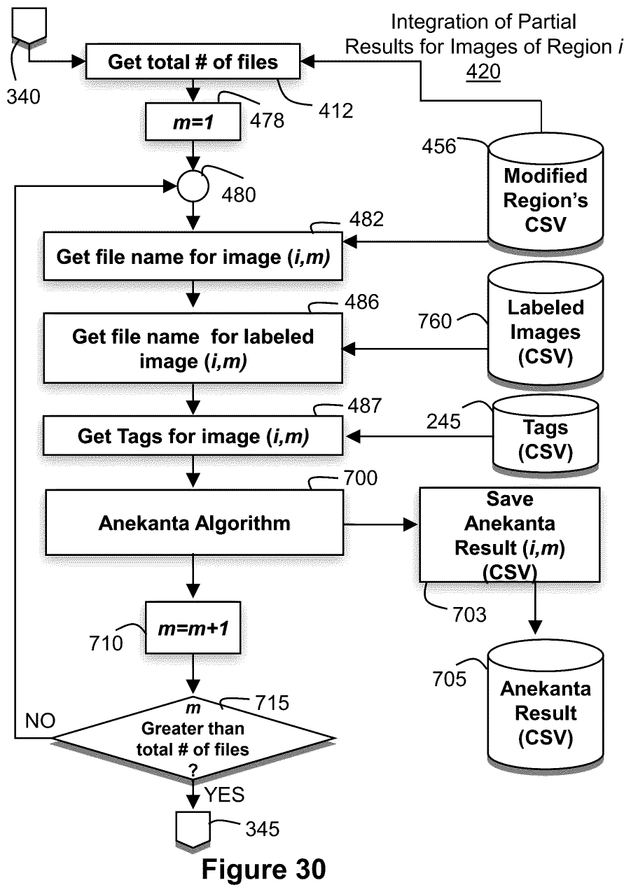

FIG. 30 is a high-level flowchart illustrating an Anekanta Algorithm-based process for integrating partial results and predictions made by a plurality of residual neural network models.

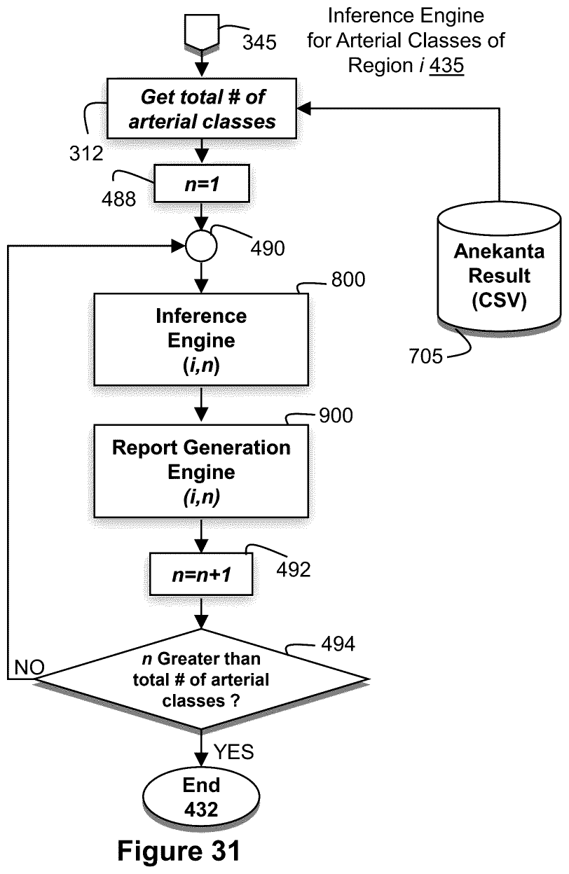

FIG. 31 is a high-level flowchart illustrating the use of an inference engine to diagnose peripheral arterial disease in a known patient, and a report generation engine to report the results.

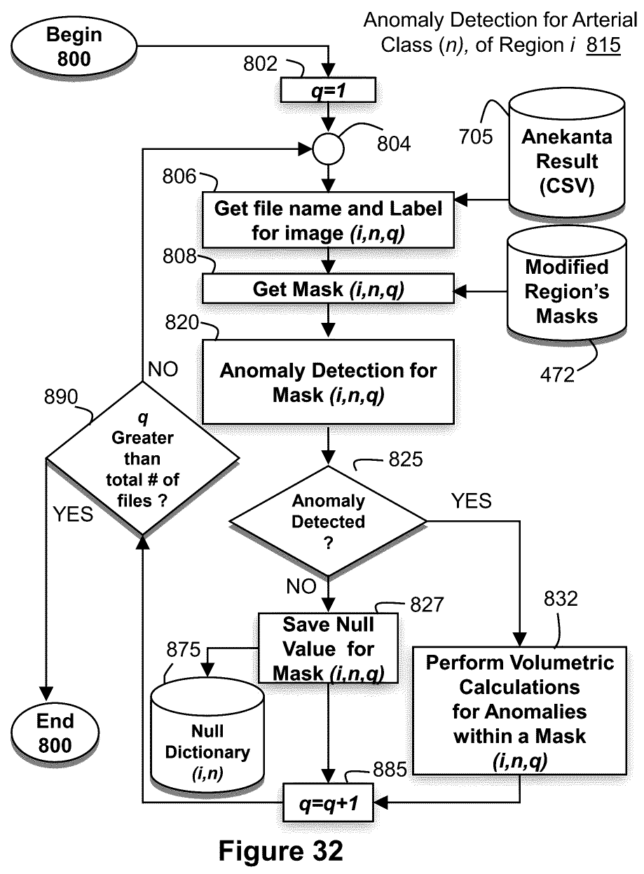

FIG. 32 is a high-level flowchart illustrating the methods of an inference engine to make diagnostic inferences regarding the presence or absence of peripheral arterial disease in a region of the peripheral arterial system of a known patient.

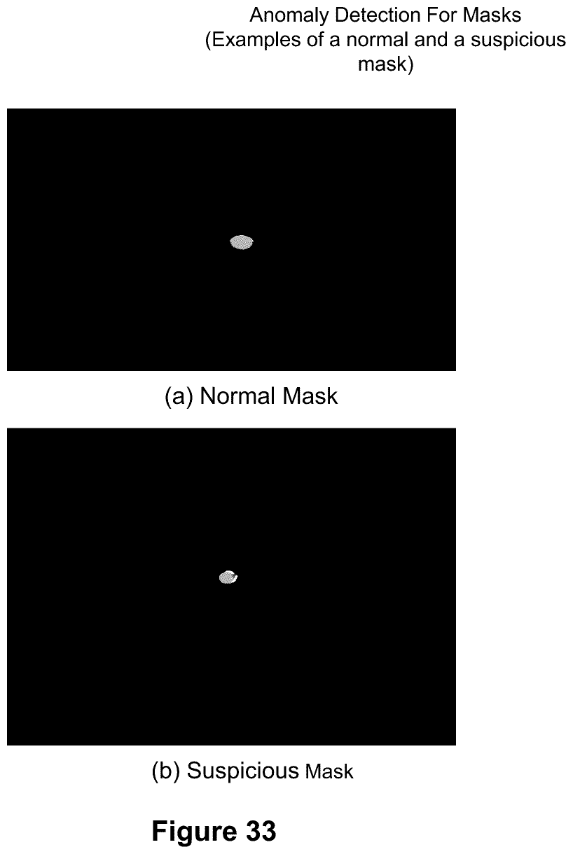

FIG. 33 illustrates a normal image mask (having a uniform grayscale pixel intensity), and a suspicious image mask with one or more anomalies (exhibiting multiple grayscale pixel intensities in it), marking the image mask as suspicious as a result of a possible disease, e.g., calcified plaque, or non-calcified plaque.

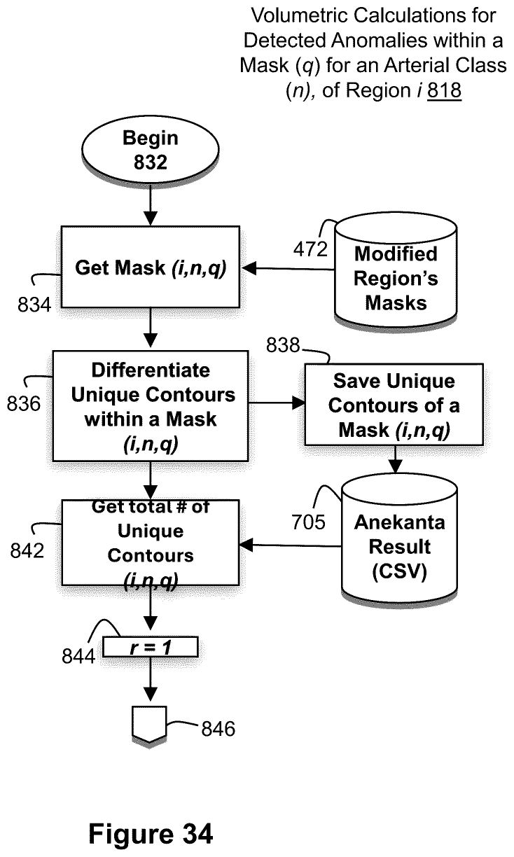

FIG. 34 is a high-level flowchart illustrating the methods for differentiating one or more contours within an image mask and determining total number of unique contours in said image mask.

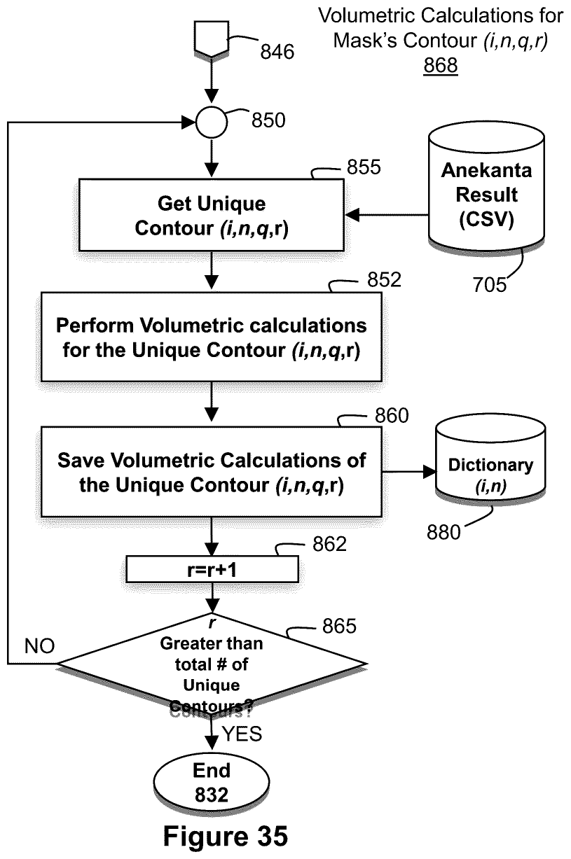

FIG. 35 is a high-level flowchart illustrating a computer-implemented iterative process for performing volumetric calculations for a contour within an image mask corresponding to an image file, in an arterial class of a region of the peripheral arterial system.

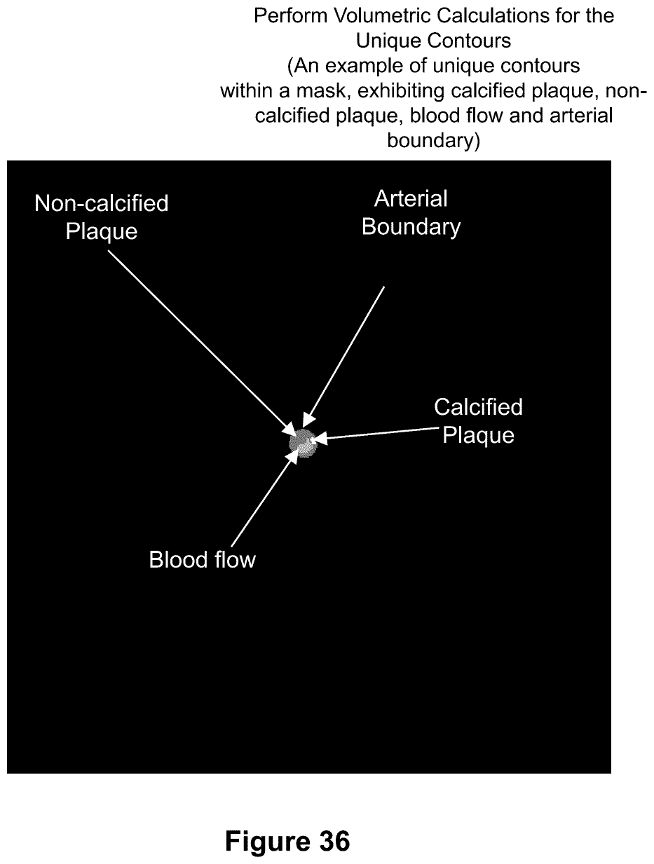

FIG. 36 illustrates multiple contours in an image mask based upon different grayscale pixel intensities within each contour.

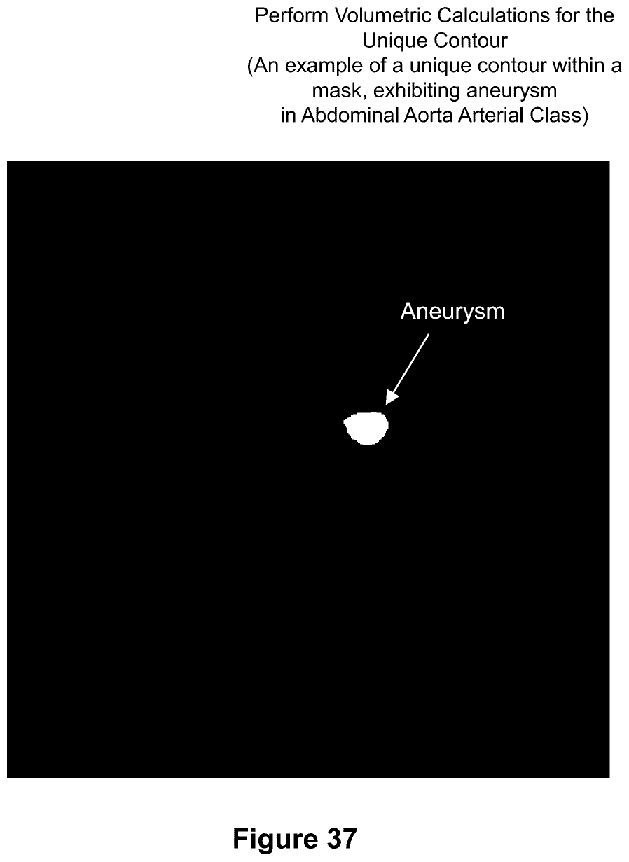

FIG. 37 illustrates an example contour that may represent the presence of aneurysm in the abdominal aorta, which may be detected by performing volumetric calculations on a contour in an image mask.

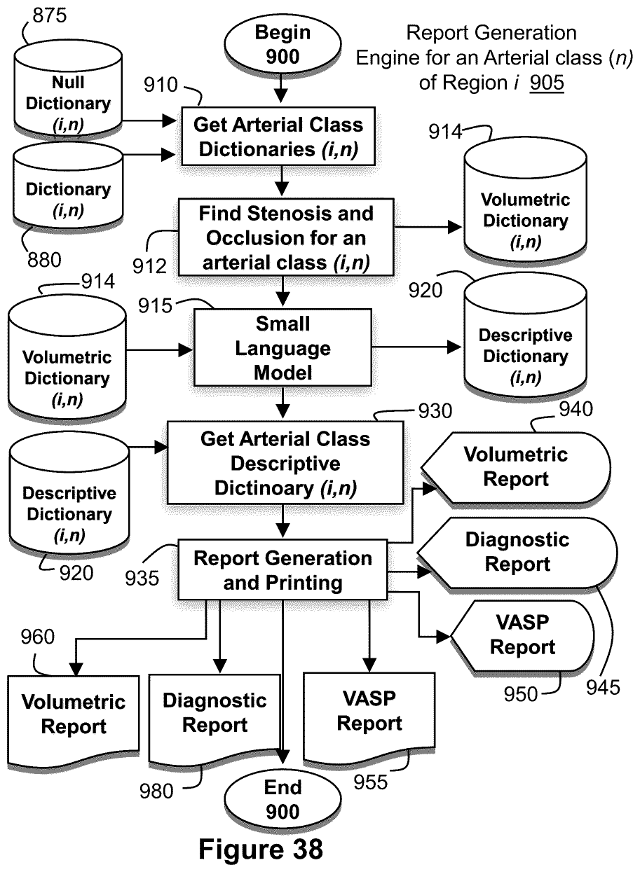

FIG. 38 is a high-level flowchart illustrating a Report Generation Engine process for reporting the presence or absence of disease in an arterial class of a region of the peripheral arterial system.

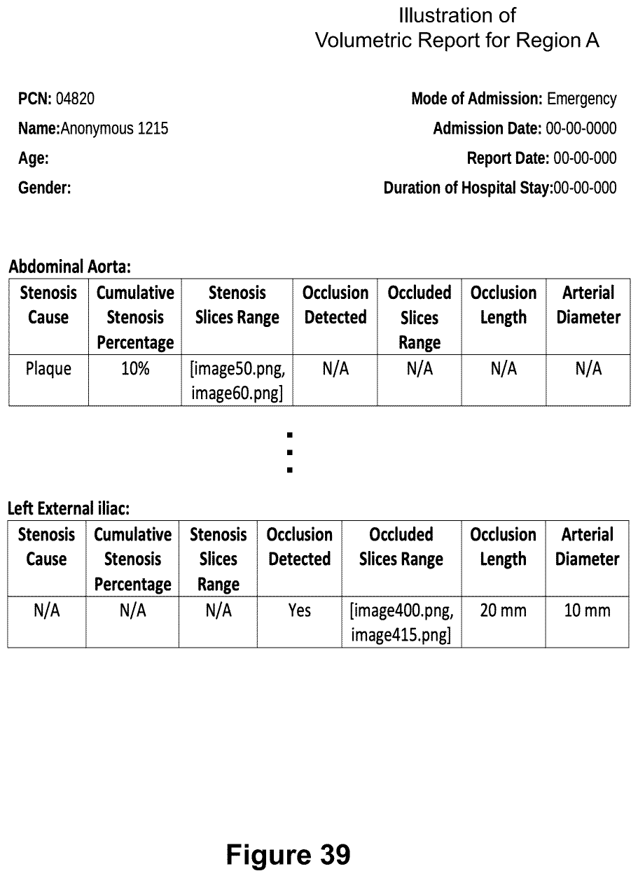

FIG. 39 illustrates an example volumetric report produced by a Report Generation Engine showing presence of peripheral arterial disease in multiple arteries.

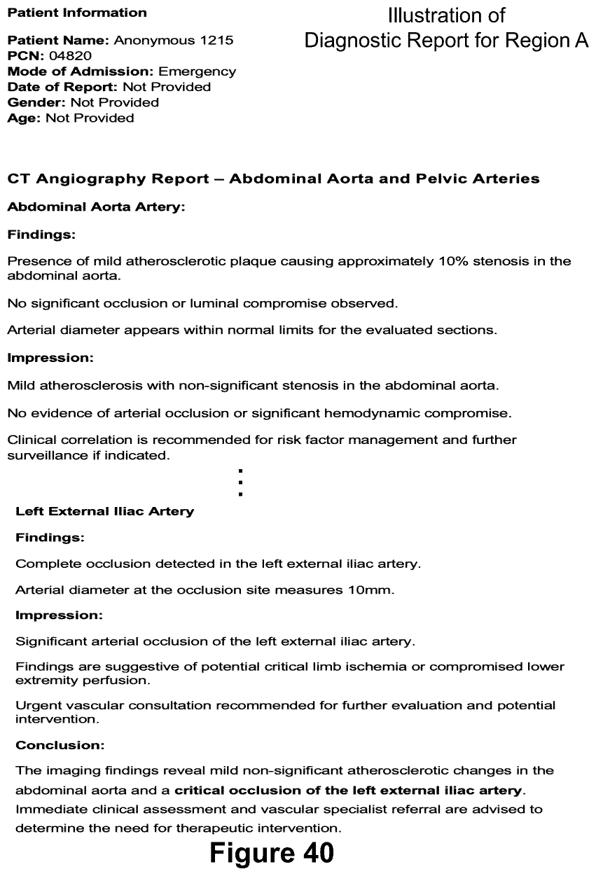

FIG. 40 illustrates an example diagnostic report produced by a Report Generation Engine showing diagnosis for a region of the peripheral arterial system.

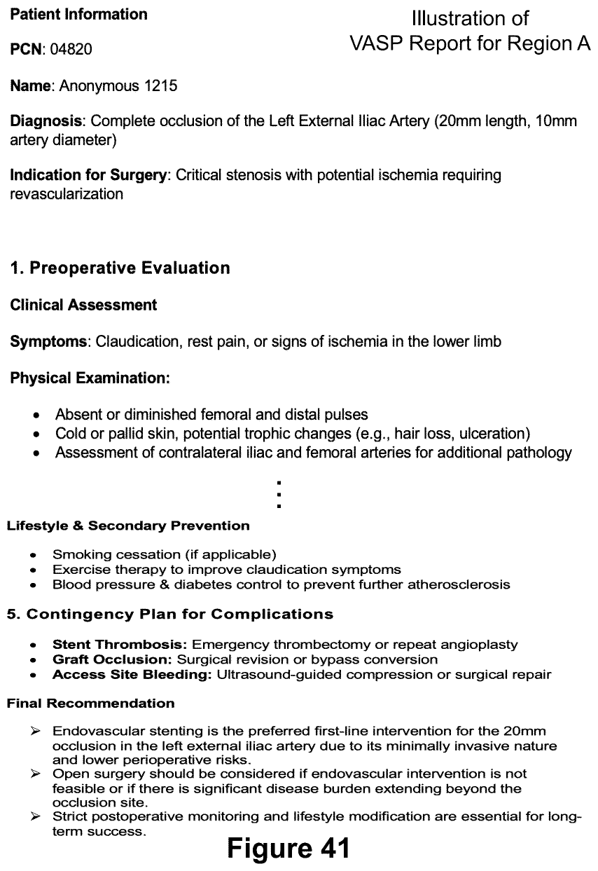

FIG. 41 illustrates an example vascular arterial surgical planning (VASP) report produced by a Report Generation Engine for a patient including a pre-operative evaluation.

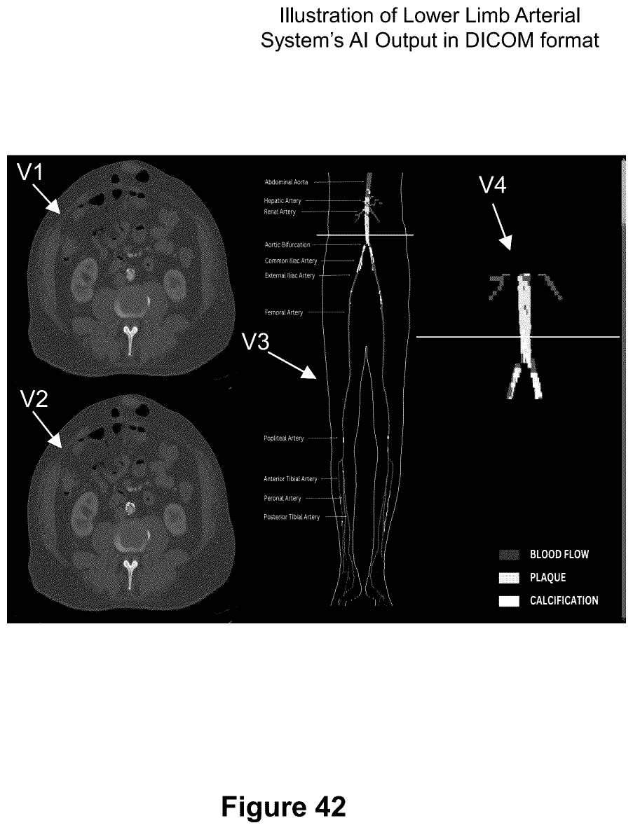

FIG. 42 illustrates an example of visualizing AI-generated output for the lower limb arterial system overlaid over the images in DICOM format.

It will be appreciated that for simplicity and/or clarity of illustration, elements illustrated in the figures have not necessarily been drawn to scale. For example, the dimensions of some of the elements may be exaggerated relative to other elements for clarity. Further, if considered appropriate, reference numerals have been repeated among the figures to indicate corresponding or analogous elements.

### DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, well-known methods and procedures have not been described in detail.

Some portions of the detailed description that follows are presented in terms of algorithms, programs and/or symbolic representations of operations on data bits or binary digital signals within a computer memory, for example. These algorithmic descriptions and/or representations may include techniques used in the data processing arts to convey the arrangement of a computer system and/or other information handling system to operate according to such programs, algorithms, and/or symbolic representations of operations.

An algorithm may be generally considered to be a self-consistent sequence of acts and/or operations leading to a desired result. These include physical manipulations of physical quantities. Usually, though not necessarily, these quantities take the form of electrical and/or magnetic signals capable of being stored, transferred, combined, compared, and/or otherwise manipulated. It may be convenient at times, principally for reasons of common usage, to refer to these signals as bits, values, elements, symbols, characters, terms, numbers and/or the like. However, these and/or similar terms may be associated with the appropriate physical quantities and are merely convenient labels applied to these quantities.

Unless specifically stated otherwise, as apparent from the following discussions, throughout the specification discussion utilizing terms such as processing, computing, calculating, determining, and/or the like, refer to the action and/or processes of a computer and/or computing system, and/or similar electronic computing device, that manipulate or transform data represented as physical, such as electronic, quantities within the registers and/or memories of the computer and/or computing system and/or similar electronic and/or computing device into other data similarly represented as physical quantities within the memories, registers and/or other such information storage, transmission and/or display devices of the computing system and/or other information handling system.

Embodiments claimed may include apparatuses for performing the operations herein. This apparatus may be specially constructed for the desired purposes, or it may comprise a general-purpose computing device selectively activated and/or configured by a program stored in the device. Such a program may be stored on a storage medium, such as, but is not limited to, any type of disk including floppy disks, optical disks, CD-ROMs, magnetic-optical disks, read-only memories (ROMs), random access memories (RAMs), electrically programmable read-only memories (EPROMs), electrically erasable and/or programmable read only memories (EEPROMs), flash memory, magnetic and/or optical cards, and/or any other type of media suitable for storing electronic instructions, and/or capable of being coupled to a system bus for a computing device and/or other information handling system.

The processes and/or displays presented herein are not inherently related to any particular computing device and/or other apparatus. Various general-purpose systems may be used with programs in accordance with the teachings herein, or a more specialized apparatus may be constructed to perform the desired method. The desired structure for a variety of these systems will appear from the description below. In addition, embodiments are not described with reference to any particular programming language. A variety of programming languages may be used to implement the teachings described herein.

Reference throughout this specification to one embodiment or an embodiment means that a particular feature, structure, or characteristic described in connection with the embodiment is included in at least one embodiment. Thus, the appearances of the phrase in one embodiment or an embodiment in various places throughout this specification are not necessarily all referring to the same embodiment. Furthermore, the particular features, structures, or characteristics may be combined in one or more embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances). For example, “about 3.5 mm” includes “3.5 mm”.

A network as referred to herein relates to infrastructure that is capable of transmitting data among nodes which are coupled to the network. For example, a network may comprise links capable of transmitting data between nodes according to one or more data transmission protocols. Such links may comprise one or more types of transmission media and/or links capable of transmitting information from a source to a destination. However, these are merely examples of a network, and the scope of the claimed subject matter is not limited in this respect.

Instructions as referred to herein relate to expressions that represent one or more logical operations. For example, instructions may be machine-readable by being interpretable by a machine for executing one or more operations on one or more data objects. However, this is merely an example of instructions, and the scope of claimed subject matter is not limited in this respect. In another example, instructions as referred to herein may relate to encoded commands which are executable by a processing circuit having a command set which includes the encoded commands. Such an instruction may be encoded in the form of a machine language understood by the processing circuit. However, these are merely examples of an instruction, and the scope of the claimed subject matter is not limited in this respect.

A storage medium as referred to herein relates to media capable of maintaining expressions which are perceivable by one or more machines. For example, a storage medium may comprise one or more storage devices for storing machine-readable instructions and/or information. Such storage devices may comprise any one of several media types including, for example, magnetic, optical or semiconductor storage media. However, these are merely examples of a storage medium, and the scope of the claimed subject matter is not limited in this respect.

Folder as referred to herein relate to allocation of space in a storage device which may be attached to a computing device and is accessible to the algorithms and computer-implemented methods of the present invention. One or more folders may be used to store input data, temporary data, and output data for an algorithm. Examples of data may include PNG images, resultant arterial images, comma-separated values (CSV) files, and mathematical/numerical results of an algorithm. A folder may be maintained in a computer's memory, or on a storage device (e.g., a hard drive), or in a cloud storage device. A folder and its contents may be deleted after their use to free up storage space. For example, if a data item in a folder has been consumed (e.g., an illustrative image or a formatted report) and is no longer needed, said data item may be deleted. Similarly, if all data stored in a folder is no longer needed, said folder may be deleted. However, this is merely an example of a folder, and the scope of the claimed subject matter is not limited in this respect.

Logic as referred to herein relates to structure for performing one or more logical operations. For example, logic may comprise circuitry which provides one or more output signals based upon one or more input signals. Such circuitry may comprise a finite state machine which receives a digital input and provides a digital output, or circuitry which provides one or more analog output signals in response to one or more analog input signals. Such circuitry may be provided in an application specific integrated circuit (ASIC) or field programmable gate array (FPGA), for example. Also, logic may comprise machine-readable instructions stored in a storage medium in combination with processing circuitry to execute such machine-readable instructions. However, these are merely examples of structures which may provide logic, and the scope of the claimed subject matter is not limited in this respect.

Unless specifically stated otherwise, as apparent from the following discussion, it is appreciated that throughout this specification discussions utilizing terms such as processing, computing, calculating, selecting, forming, enabling, inhibiting, identifying, initiating, receiving, transmitting, determining and/or the like refer to the actions and/or processes that may be performed by a computing platform, such as a computer or a similar electronic computing device, that manipulates and/or transforms data represented as physical electronic and/or magnetic quantities and/or other physical quantities within the computing platform's processors, memories, registers, and/or other information storage, transmission, reception and/or display devices. Further, unless specifically stated otherwise, process described herein, with reference to flow diagrams or otherwise, may also be executed and/or controlled, in whole or in part, by such a computing platform.

Methods of the present invention may be implemented in a single computer system, or in a client-server configuration, or in a network-based system, or in a cloud-based system configuration, or any combination thereof. In all computer configurations listed above that may use a network, a server may communicate with one or more client computers over a network. A client computer may store data either locally and/or on a server, and access all remote data via the network. A client computer may transmit requests for data, and/or requests for online services, to the server via the network. The server may perform requested services and provide data to the client computer(s). The server may also transmit data adapted to cause a client computer to perform a specified function, e.g., to perform a calculation, to display specified data on a screen, etc. Certain steps of the methods being described may be performed by a server and/or by other computers/processors in the network-based systems including cloud-computing systems. The methods being described may make use of general purpose central processing units (CPUs), and/or graphic processing units (GPUs), and/or computer hardware/software systems specialized for running neural networks for training a plurality of artificial-intelligence based neural network models to learn medical image analysis from the angiography scans of a large population of anonymized patients, using said trained models to analyze medical images of a known patient, and reporting and/or visualizing diagnostic predictions for use by medical researchers and clinicians.

Image File Format as referred to herein relate to a file format for receiving, displaying, processing, storing, and communicating images in a compressed or uncompressed form. Image data compression process may use a lossy compression algorithm or a lossless compression algorithm. Raster image formats represent 2D images. Size of an image file generally depends upon the number of pixels in the image and color depth (bits per pixel). Image formats may include PNG (Portable Network Graphics), JPEG (Joint Photographic Expert Group), GIF (Graphics Interchange Format), BMP (Microsoft Windows bitmap file format), and SVG (Scalable Vector Graphics). However, this is merely an example of an image file format, and the scope of the claimed subject matter is not limited in this respect.

Medical Imaging as referred to herein relate to any imaging technique for scanning medical images of a patient including computed tomography (CT) or contrast-enhanced computed tomography angiography (CTA). CT scans are also known as computed axial tomography (CAT) scans. Contrast-enhanced computed tomography angiography (CTA) scans are also known as Contrast CT scans. Volume rendering techniques may be used in computer software to produce 3D images by combining a plurality of 2D images. Image manipulation software including 3D visualization tools enable radiologists and physicians to view important structures in a patient's body in greater detail for diagnosing and treating a disease. However, this is merely an example of medical imaging, and the scope of the claimed subject matter is not limited in this respect.

Computed tomography scan (CT scan) as referred to herein relate to detailed images of a body obtained using a CT scanner for medical imaging. CT scanners may use a rotating X-ray tube with a row of detectors to measure attenuation of X-ray by different body tissues. Multiple measurements may be taken from different angles, relative to the body being scanned, and are processed on a computer to produce cross-sectional images of a body. However, this is merely an example of a CT/CAT scan, and the scope of the claimed subject matter is not limited in this respect.

Contrast-enhanced computed tomography angiography (CTA) or Contrast CT scans as referred to herein relate to visualizing arteries and veins throughout the body including brain, lungs, kidneys, arms and legs. Radiocontrasts (e.g., iodine-based contrasts) may be used while making contrast CT or CTA scans to highlight blood vessels and delineate them from surrounding tissue. However, this is merely an example of a contrast CT or CTA scan, and the scope of the claimed subject matter is not limited in this respect.

Angiograms as referred to herein relate to Computed tomography (CT) scans, computed axial tomography (CAT) scans, Contrast CT scans, or Contrast-enhanced computed tomography angiography (CTA) scans in one or more embodiments of the present invention. However, this is merely an example of angiograms, and the scope of the claimed subject matter is not limited in this respect.

Instance number as referred to herein relate to a DICOM standard attribute that is assigned to each image instance in a DICOM database. A DICOM system may assign a unique integer identifier as an attribute to each scanned image instance of a patient in a DICOM database to facilitate the storage and retrieval of said image instance by a computer system. However, this is merely an example of an instance number attribute, and the scope of the claimed subject matter is not limited in this respect.

Multiplanar Reformatted Imaging as referred to herein relate to visualizing CT scan images data in transverse (or axial), coronal, or sagittal plane. Axial plane is an anatomical plane that divides the body into superior (upper section) and inferior (lower section). Coronal plane divides the body into dorsal (back section) and ventral (front section). Sagittal plane divides the body into left and right sections. Axial view images may be referred to as the “primary” (or original) images. One or more derived image views (e.g., coronal and/or sagittal plane view) may be derived from the “primary” images using specialized tools provided by the imaging machine vendor. During the scanning process, orientation of a patient in a CT machine depends on the diagnosis being performed. For example, patient is laid feet first for capturing lower limb scans. For scanning upper limbs or head, patient is laid head first. While performing a CT scan of a patient, technician assigns “L” (left) and “R” (right) labels which appear in DICOM images. Radiologist uses these labels in the DICOM viewer to accurately identify the left and right sides of a patient in images. When a radiologist views an axial plane view image while sitting in front of a DICOM monitor, left side of the image corresponds to patient's right-hand side and right side of the image corresponds to patient's left side. However, these are merely examples of Multiplanar Reformatted Imaging, and the scope of the claimed subject matter is not limited in this respect.

Axial view (also known as axial plane view or horizontal view) as referred to herein relate to visualizing CT scan image data in horizontal slices that divide the body into superior (upper section) and inferior (lower section). Axial plane may also be referred to as transverse plane, horizontal plane, transaxial plane, or simply as a cross-section. However, this is merely an example of axial view, and the scope of the claimed subject matter is not limited in this respect.

Hounsfield scale as referred to herein relate to a quantitative scale for describing radiodensity in medical CT imaging. The Hounsfield unit (HU) scale is a linear transformation of the original linear attenuation coefficient measurement. The radiodensity of air is defined as −1,000 HU and water is defined as 0 in Hounsfield units (HU). Tissues and other structures in a human body that absorb more x-rays have higher HU values. Examples of radiodensity include human fat (−120 to −90 HU), unclotted blood (+13 to +50 HU), clotted blood (+50 to +75), and soft tissue (+100 to +300 HU). Cancellous bone is composed of spongy, porous, bone tissue filled with red bone marrow. Radiodensity of Cancellous bone is approximately +700 HU. Cortical bone is approximately +1,900 HU as it is highly dense—it makes up the shells and shafts of long bones as well as shells of short, flat, and irregular bones. The HU value for metals is over +3,000. The HU values for each pixel in a CT scan are converted into a digital image by assigning a gray scale intensity to each value. Air with an assigned value of −1,000 HU is shown as black in images. The higher the number, the brighter/whiter the pixel intensity in grayscale image. Therefore, human flesh, arteries, organs, bones and other structures appear in different shades of white on grayscale in a DICOM image depending upon their radiodensity. However, this is merely an example of Hounsfield scale, and the scope of the claimed subject matter is not limited in this respect.

Windowing protocol as referred to herein relate to image visualization and image conversion technique including selection of a window (i.e., a range) of HU values while visualizing and/or processing an image. List of windowing protocols may include soft tissue window, lung window, Mediastinum window, and bone window protocol. In a windowing protocol, HU values of a window help in highlighting or suppressing some of the details in an image. For example, Bone windowing protocol is useful for highlighting bones and calcification in arteries, whereas Mediastinum windowing protocol is used for visualizing soft tissue organs. The arterial system itself is soft-tissue organ and to accurately determine the outer diameter of an artery, “Mediastinum windowing” protocol is used to highlight soft tissue organs, suppressing dense organs for better visualization. The outer boundary of an artery is visually confirmed in a side-by-side comparison of Bone Windowing and Mediastinum Windowing images. However, these are merely some examples of windowing protocols and how they are used, and the scope of the claimed subject matter is not limited in this respect.

Bone windowing protocol as referred to herein relate to a windowing protocol that may be used for better visualization of the arterial system while suppressing surrounding soft tissue and empty spaces. It may have a minimum value of −500 HU and a maximum value of +1,300 HU. It is more effective in highlighting high-density structures (e.g., bones and calcified plaque in arteries), making it valuable for detecting cases of peripheral arterial disease in patients. This protocol provides clear visualization and high contrast of features, enabling the detection of finer details related to arterial blockages or stenosis, particularly in advanced cases of peripheral arterial disease where calcifications are prevalent. While plaque does not shine through like bones or calcifications, it does appear as a dense and dark area within the arterial boundaries, making it recognizable. However, this is merely an example of bone windowing protocol, and the scope of the claimed subject matter is not limited in this respect.

Peripheral Arterial System 180 as referred to herein encompasses the network of arteries that supply blood to the peripheral regions of the body, including the upper limbs, chest, abdomen, and lower limbs as shown in FIG. 2. This system is critical for delivering oxygenated blood from the heart to all regions of the body, excluding few areas like the brain and heart. The Peripheral Arterial System includes major arteries, e.g., subclavian, axillary, brachial, radial artery, ulnar artery; aorta and its branches such as hepatic artery, renal arteries, splenic artery and the pulmonary veins (oxygenated blood vessels); common iliac arteries, external iliac arteries, femoral, popliteal, Anterior tibial arteries, Posterior tibial arteries and their ancillary branches. However, this is merely an example of how Peripheral Arterial System may be defined, and the scope of the claimed subject matter is not limited in this respect.

Region of the Peripheral Arterial System as referred to herein may be defined either from a system design perspective or from a clinical perspective. List of main regions of the Peripheral Arterial System may include Brachioaxillary Arterial Region, Radioulnar Arterial Region, Aortoiliac and Visceral Arterial Region, Femoropopliteal Region, and Crural Region. Defining a region may help in performing one or more radiological procedures that are required for making a clinical diagnosis. For example, a clinician may prescribe an abdominal scanning procedure for a patient, covering only the aorta. In another situation, a clinician may prescribe a procedure for scanning lower limbs only while covering aorta, common iliac and femoral arteries (above knee). A clinician may, in another example, prescribe a procedure for scanning the entirety of lower limb, covering aorta and all of the arteries down to the digits of the feet. Similarly, regions and their sub-regions may be defined for the upper body arterial system. However, this is merely an example of how a region of peripheral arterial system may be defined, and the scope of the claimed subject matter is not limited in this respect.

Brachioaxillary Arterial Region as referred to herein comprises a region of the upper limb arterial system which includes thoracic aorta, carotid, subclavian, axillary, and brachial arteries up to the elbow. This region is essential for supplying blood to the shoulder, arm, and upper portions of the upper limb. The Brachioaxillary Arterial Region plays a critical role in the vascular network of the upper extremity, delivering blood to the muscles, skin, and bones of the arm. However, this is merely an example of how Brachioaxillary Arterial Region of peripheral arterial system may be defined, and the scope of the claimed subject matter is not limited in this respect.

Radioulnar Arterial Region as referred to herein comprises a region of the arterial system from the elbow to the wrist which includes radial and ulnar arteries that supply blood to the forearm. This region is vital for distributing blood to the muscles, tendons, and tissues of the forearm, excluding the palm and hand. The Radioulnar Arterial Region is significant in supporting forearm functionality by ensuring adequate blood flow to both the lateral and medial parts of the forearm. However, this is merely an example of how Radioulnar Arterial Region of peripheral arterial system may be defined, and the scope of the claimed subject matter is not limited in this respect.

Aortoiliac and Visceral Arterial Region as referred to herein comprises a region of the arterial system which includes abdominal aorta, common iliac arteries, external iliac arteries, as well as the major arteries branching off from aorta that supply blood to liver, kidneys, spleen and lungs. This includes the hepatic artery, renal arteries, splenic artery, and pulmonary veins (oxygenated blood vessels). These arteries are critical for delivering oxygenated blood from the aorta to the lower extremities and vital organs. The Aortoiliac and Visceral Arterial Region plays a vital role in the vascular network, providing both primary pathways to the lower limbs and essential organs. However, this is merely an example of how Aortoiliac and Visceral Arterial Region of peripheral arterial system may be defined, and the scope of the claimed subject matter is not limited in this respect.

Femoropopliteal Region as referred to herein comprises a region of the arterial system which extends from femoral arteries down to popliteal arteries. This region is crucial for supplying blood to the thigh and knee areas. The Femoropopliteal Region, as a vascular network of the lower limb, facilitates the flow of blood through major arteries that support the upper leg and knee. However, this is merely an example of how Femoropopliteal Region of peripheral arterial system may be defined, and the scope of the claimed subject matter is not limited in this respect.

Crural Region as referred to herein comprises a region of the arterial system from the popliteal arteries down to the arteries of the foot. This region is vital for supplying blood to the lower leg, feet, and digits of the feet. The Crural Region includes peroneal, anterior tibial, posterior tibial arteries, and their ancillary branches. However, this is merely an example of how Crural Region of peripheral arterial system may be defined, and the scope of the claimed subject matter is not limited in this respect.

Arterial class as referred to herein is a named set comprising of one or more members of the peripheral arterial system in it. Each member of an arterial class may be another named arterial class or a named artery. For example, abdominal aorta is an arterial class in the Aortoiliac and Visceral Arterial Region that may include a plurality of members (or branches), e.g., hepatic artery, a class of renal arteries, splenic artery. Few examples of arterial classes that may be stored in separate folders are illustrated in FIG. 9 (abdominal aorta), FIG. 10 (common iliac arteries), and FIG. 11 (external iliac arteries). However, this is merely an example of how an arterial class may be defined, and the scope of the claimed subject matter is not limited in this respect.

Arterial Stenosis as referred to herein relate to the narrowing in an artery (member of an arterial class), which may restrict the blood flow. This narrowing may be caused by the buildup of substances like non-calcified plaque (a mix of fat, cholesterol, and other materials) and/or calcified plaque (calcium deposits) along the arterial walls, or both. However, this is merely an example of how the arterial stenosis may be defined, and the scope of the claimed subject matter is not limited in this respect.

Arterial occlusion as referred to herein relate to complete blockage in an artery (member of an arterial class), which may significantly and/or entirely impede blood flow. This blockage may be caused by the accumulation of non-calcified plaque (a mix of fat, cholesterol, and other substances), thrombus (blood clot), or embolus (a traveling clot or debris and/or calcified plaque (calcium deposits) along the arterial walls or both. However, this is merely an example of how arterial occlusion may be defined, and the scope of the claimed subject matter is not limited in this respect.

Neural network as referred to herein relate to an artificial intelligence-based computer model. A neural network model may comprise of many nodes wherein each node may be connected to other nodes using one or more edges or connections. Each node may receive signals from its connected nodes, process them and send a signal to other connected nodes. Output of each node may be computed using an activation function (a non-linear function) of the sum of its inputs. Strength of the signal at each connection may be determined by a weight which is adjusted during the learning process. Types of neural networks as referred to herein may include convolutional neural networks (CNNs), recurrent neural networks (RNNs), generative adversarial neural networks (GANs), deep learning neural networks and transformer-based neural networks. However, this is merely an example of a neural network, and the scope of the claimed subject matter is not limited in this respect.

Neural network layer as referred to herein relate to collection of nodes in a neural network model (also known as a neural net model). Signals may travel from the first layer (input layer) to the last layer (output layer) while traversing one or more intermediate layers. However, this is merely an example of a neural network layer, and the scope of the claimed subject matter is not limited in this respect.

Convolution as referred to herein relate to a mathematical operation on two functions that results in a third function and is defined as the integral of the product of the two functions after one is flipped and shifted. Assume f (x) and g (x) are two different functions. Function g (x) after reflection about the y-axis results in g (−x). Shifting the reflected function g (−x) by a value t results in g (−x+t) which can also be written as g (t−x). The product of the function f (x) and the shifted, reflected function g (t−x) is integrated over all values of x, resulting in the convolution of f and g, formally expressed as:

$$( f * g ) ( t ) = \int f ( x ) * g ( t - x ) dx$$

In image processing applications, a convolution often involves a smaller “kernel” (also known as a “filter”) that is slid over a larger input image. There are multiple types of convolutions, e.g., Pointwise, Depthwise, Depthwise separable, and Standard (Full). Dimension of an input image includes its height (in pixels), width (in pixels), and number of input channels. Number of input channels for a color image are 3 for RGB (red, green, and blue colors) whereas a grayscale images has only one channel. After applying a set of filters in a convolution, each filter produces one output channel. A 1×1 convolution is a pointwise convolution where the filter (i.e., kernel) has a spatial size of 1×1 (only one pixel across all channels). Therefore, a 1×1 convolution combines information only across channels without changing spatial dimensions. A 3×3 convolution is a convolution operation where the filter has a spatial size of 3×3. A full 3×3 convolution mixes channels and spatial information for full feature extraction. A 3×3 convolutional filter slides over the input image like a 3×3 window. At each position of this window, a single value dot product between the filter and the 3×3 patch of the input is computed. These dot products are collected in a 2D grid and are known as a feature map. However, this is merely an example of convolution as a mathematical operation, and the scope of the claimed subject matter is not limited in this respect.

Convolutional neural network (CNNs) as referred to herein relate to a neural network developed using a deep learning algorithm optimized for tasks requiring object recognition including image classification, detection, and segmentation, by learning features through filters (or kernels) and convolutional operations. A CNN comprises a plurality of neural network layers including convolutional layers, pooling layers, fully connected layers, and an output layer. Convolutional and pooling layers perform feature extraction, while fully connected layers map the extracted features into final output. A CNN generates feature maps by convolving filters over the input image to serve as input for subsequent layers in the neural network for learning higher-level features. Features become progressively more complex as data moves through the network layers, with early layers focusing on simple features (like edges) and later layers identifying more complex features (like objects). However, this is merely an example of a convolutional neural network (CNN), and the scope of the claimed subject matter is not limited in this respect.

Residual neural network as referred to herein relate to a convolutional neural network (CNN) which is configured with some skip connections to bypass some layers in order to overcome the vanishing gradient problem associated with deep CNNs that simply feed the output of a node to another node in the next layer. Residual neural networks learn a “residual” function using the difference between the input and the desired output. A shortcut path for data flow is created by adding the residual function to the original input. Without deploying said skip connections, gradients guiding the adjustment of network's weights during the training process may become extremely small as they propagate backward through the layers, making it difficult for the earlier layers to learn effectively. As a result of using skip connections to bypass some layers, a Residual neural network may also overcome the problem of exploding gradient that may occur when the gradients guiding the adjustment of network's weights during the training process become extremely large as they propagate backward through the layers, making it difficult for the earlier layers to learn effectively. However, this is merely an example of a Residual neural network, and the scope of the claimed subject matter is not limited in this respect.

Region-based Convolutional Neural Network (R-CNN) as referred to herein relate to a family of machine learning models for detecting and categorizing one or more objects present in an image. Input image goes through a pre-processing pipeline to generate proposals for objects present in different bounding boxes within the input image. R-CNN may produce zero or more bounding boxes in an image wherein each bounding box (also known as a region of interest in computer vision terminology) may contain an object in it. R-CNN searches for and extracts zero or more regions of interest (ROIs) in an image wherein each ROI is a rectangle (i.e., a bounding box) representing the boundary of an object in it. Each proposal may be resized and processed by the R-CNN for feature extraction. Features corresponding to each proposal may then be used to infer each object's presence and class of interest from the Support Vector Machines (SVMs) classifiers. Finally, the bounding box regressor fine-tunes the locations of the objects. ROls are fed to one or more convolutional neural networks to recognize and categorize the object in it. Types of R-CNNs as referred to herein may include Fast R-CNN, Faster R-CNN, and Mask R-CNN. However, this is merely an example of a region-based convolutional neural network (R-CNN), and the scope of the claimed subject matter is not limited in this respect.

Faster R-CNN with a Feature Pyramid Network (FPN) as referred to herein relate to the use of a two-stage object detection algorithm for identifying and locating objects in an input image. Faster R-CNN with a Feature Pyramid Network (FPN) comprises: bottom-up pathway, top-down pathway, lateral connections between the bottom-up and top-down pathways, a region proposal network (RPN), a region of interest (ROI) pooling layer, and two parallel fully connected layers: a classification head layer, and a bounding box regression head layer that further refines the coordinates of the detected objects. For the purpose of extracting feature maps, the FPN subsystem (in a Faster R-CNN) comprises two types of neural network pathways (bottom-up and top-down), and lateral connections between the two pathways. The bottom-up layers of the FPN serve as a backbone network that may be a pre-trained residual neural network for extracting a pyramid of feature maps from an input image. The bottom-up pathway of the FPN may comprise a plurality of convolution modules wherein each convolution modules (or a stage) has one or more convolution layers in it. Spatial dimension is reduced in half (½) by doubling the stride at each stage as feature maps moves up through the bottom-up pathway. In other words, feature maps are down sampled at each stage by doubling the stride. Therefore, the feature maps obtained at lower levels in the bottom-up pathway have more details in them whereas the feature maps at upper layers have more semantic information. The output (Ci) of each convolution module i (in the bottom-up pathway of the FPN) is later used in the top-down layers of the FPN. A 1×1 convolution is applied to the output of the topmost convolution module (in the bottom-up pathway) prior to feeding it to the topmost module of the top-down pathway which passes it on to an RPN (region proposal network) for predicting objects present in the top stage feature maps. At each stage, while moving down through the top-down pathway, results of the previous stage (in the top-down pathway) are upsampled by 2 with the help of a nearest neighbor upsampling method. Resulting upsampled feature maps for this stage of the top-down pathways are merged with the feature maps from the bottom up pathway after applying a 1×1 convolution to the bottom-up feature maps. A 3×3 convolution is applied to the results of the merge operation before feeding them to the RPN for predicting objects present in that stage. RPN is a fully convolutional network that simultaneously predicts object boundaries and objectness scores at each location. The RPN generates region proposals by sliding a small network over the feature map. At each location of the sliding window, RPN generates one or more region of interest (ROI) proposals each with a classification score indicating the likelihood of the presence of an object in the input image. Region of interest (ROI) pooling layer produces a fixed-size feature map for each proposal. A classification head layer predicts the class of the object in each region proposal and a bounding box regression head layer further refines the coordinates of the detected object for each proposal. However, this is merely an example of a Faster region-based convolutional neural network (Faster R-CNN) with a Feature Pyramid Network (FPN), and the scope of the claimed subject matter is not limited in this respect.

Training data as referred to herein relate to real-world patient data that may have been, anonymized (to conform with HIPAA rules), labeled, annotated and structured prior to its use during the learning process of a neural network model. HIPPA (Health Insurance Portability and Accountability Act of 1996) is a US federal law and a national standard for protecting sensitive patient health information from disclosure without the patient's consent or knowledge. Neural network's structure and its parameters may be further tweaked by iteratively updating its parameters to minimize a defined loss function. However, this is merely an example of training data, and the scope of the claimed subject matter is not limited in this respect.

Test data as referred to herein relate to real-world patient data that may have been, anonymized (to conform with HIPAA rules), labeled, annotated and structured prior to its use in a testing environment to test the predictions being made by a neural network model after each learning phase is over. Neural network's structure and its parameters may be further tweaked by iteratively updating its parameters to minimize a defined loss function. However, this is merely an example of test data, and the scope of the claimed subject matter is not limited in this respect.

Known patient as referred to herein relate to any patient that has been CT scanned for clinical reasons. In an embodiment, a known patient may not be a part of the annonymized patients whose images data was previously used to train a plurality of neural network models. However, this is merely an example of a known patient, and the scope of the claimed subject matter is not limited in this respect.

Known patient data as referred to herein relate to a real-world known patient's data also known as unseen data, i.e., data that was not part of either the training data or test data. Known patient data may not be anonymized and may be structured for feeding into a plurality of neural network models (which have been trained previously with the help of training and test data sets) for making predictions to assist with a possible diagnosis of a known patient's disease or its progress. However, this is merely an example of a known patient data, and the scope of the claimed subject matter is not limited in this respect.

Database as referred to herein relate to a system for storing and retrieving data for processing by one or more computer-implemented methods. A database may be any type of database including SQL, non-SQL, hierarchical, and/or any other type of database that is suitable for accessing and processing data. A database may use a naming convention for the database keys to help in storing and retrieving data. A database key is a field or a set of fields that uniquely identify a record in the database. However, this is merely an example of a database, and the scope of the claimed subject matter is not limited in this respect.

File System (or filesystem) as referred to herein relate to a system for storing and retrieving data files for processing by one or more computer-implemented methods. A filesystem may use a hierarchy of folders for organizing files in them. A folder naming convention may be used to facilitate access to the files in each folder in a filesystem. Similarly, a file naming convention may be used to facilitate the storage and retrieval of a file in a folder. For example, a concatenation of a patient id (ID) with a DICOM image instance number may be used as a name for a file in a file naming convention to make the storage and retrieval of a file easier for one or more computer-implemented methods. However, this is merely an example of a file system, and the scope of the claimed subject matter is not limited in this respect.

Learning process as referred to herein may relate to methods for optimizing neural network's parameters to minimize the difference between the predicted output and the actual target values in anonymized training data and test data. For convolutional neural networks, back-propagation is one of the gradient-based methods that may be used to estimate the parameters of the network. Neural network may learn from labeled data by iteratively updating its parameters to minimize a defined loss function. After a neural network has been trained, it may be used for predicting outputs in response to new inputs of known patients. However, this is merely an example of learning process of a neural network, and the scope of the claimed subject matter is not limited in this respect.

Recognition as referred to herein relate to the methods of machine learning that identify one or more objects that may be present in an image. In general, a recognition method may identify individual objects in an image without categorizing them. For example, a recognition method may identify the presence of arteries within an image, distinguishing them from surrounding tissues, organs and bones. Similarly, in an image taken on a hiking trail, a recognition method may identify hikers (distinguishing them from the surrounding scenery including trees, boulders and sky) without categorizing each hiker object as an adult or a child. However, this is merely an example of a recognition method, and the scope of the claimed subject matter is not limited in this respect.

Classification as referred to herein relate to the methods of machine learning that assign a category to an object that may have been identified by a recognition method. After objects have been recognized, a classification method may assign each object to a specific category. For example, a classification method may categorize different arteries based on their location or characteristics (e.g., coronary artery, carotid artery, femoral artery). However, this is merely an example of a classification method, and the scope of the claimed subject matter is not limited in this respect.

Segmentation as referred to herein relate to the methods of machine learning that outline the exact pixels that may belong to each object (which has been previously identified using a recognition method and categorized using a classification method), and creating a detailed ‘mask’ of each object. For example, machine learning may recognize a car in a street scene, classify it as a ‘sedan’ and then segment the car pixels from the background to precisely outline its shape and location in the image. In another example, a segmentation method may be used to accurately outline the boundaries of each artery in an image, creating a detailed digital representation of the vessel structure. However, this is merely an example of a segmentation method, and the scope of the claimed subject matter is not limited in this respect.

The term Bitwise AND Operation as referred to herein relate to a pixel-wise logical conjunction that retains only the areas where both the image and the mask have nonzero values. This operation effectively isolates specific areas of interest within the image by masking out unwanted areas, ensuring that only the overlapping portions between the image and mask remain visible. The isolated area of interest within the image also described as Gray Mask. However this is merely an example of how to use Bitwise AND Operation to isolate objects using their masks within an image, the scope of the claimed subject matter is not limited in this respect.

Resultant Arterial Image as referred to herein relate to an output of Bitwise AND Operation method on an input image (captured during the arterial phase of a CT scan and converted into a 2D PNG image format) and its corresponding mask. A resultant arterial image may be standardized to a dimension of 512×512 pixels for its height and width by adjusting an image's pixel array. Areas where both the image and the mask have nonzero pixel values shall be visible, ensuring that only the overlapping portions between the image and the mask remain visible, as illustrated in FIG. 21. However, this is merely an example of how a Resultant Arterial image may be defined, and the scope of the claimed subject matter is not limited in this respect.

Artery Labeling as referred to herein relates to the methods for use in machine learning that assigns a label and corresponding bounding box coordinates to each artery present in an image. After an arterial class has been classified, an artery labeling method may assign a label and bounding box coordinates to each artery (member of an arterial class) present in an image. For example, abdominal aorta is an arterial class in the Aortoiliac and Visceral Arterial Region that may include a plurality of members (or branches), e.g., hepatic artery, renal arteries, and splenic artery. However, this is merely an example of how Artery Labeling may be defined, and the scope of the claimed subject matter is not limited in this respect.

Anekanta Algorithm as referred to herein relate to a method of machine learning for achieving an integrated prediction by combining results from a plurality of base models wherein each base model may have provided only a partial prediction. A base model as referred to herein relate to any artificial intelligence model whose results may be fed to the Anekanta Algorithm for machine learning. The Anekanta Algorithm may focus on how a plurality of partial predictions, made by one or more computer-implemented methods, may support each other, leading to an integrated prediction using a set of rules. However, this is merely an example of the Anekanta Algorithm, and the scope of the claimed subject matter is not limited in this respect.

Inference engine as referred to herein relate to a computer-implemented method of applying a set of rules embodied in an expert system to process data it may receive to diagnose the presence or absence of disease in an arterial class of a region of the peripheral arterial system. However, this is merely an example of an inference engine, and the scope of the claimed subject matter is not limited in this respect.

FIG. 1 illustrates a traditional system for diagnosing disease using angiograms of a patient. As shown in FIG. 1, an image acquisition device 105, controlled by an image acquisition software 115, captures CT/CAT/CTA scan angiogram images of a patient. Angiogram images are communicated to an image manipulation software 120 either locally or over a communications network 110. Angiogram images may also be reproduced in a hard copy form (radiographs) 125 and/or saved in a Picture Archiving and Communication System (PACS) 135 using an international standard format known as Digital Imaging and Communications in Medicine (DICOM). A human radiologist 155 examines a patient's angiogram images with the help of a DICOM viewer software 145 to diagnose possible disease in Peripheral Arterial System 180 (illustrated in FIG. 2). Human radiologist 155 may also document the results of his/her diagnosis and make them a part of patient's electronic medical record (EMR) 165.

Referring now to FIG. 3, in a preferred embodiment, a high-level block diagram 200 shall be discussed, illustrating training a plurality of neural network models in accordance with one or more embodiments of the present invention. In an embodiment, a network 110 may be used for data communication between blocks shown in the high-level block diagram 200. In one or more embodiments, network 110 may comprise of Internet, and the scope of the claimed subject matter is not limited in this respect.

In one or more embodiments of the present invention, CTA scans or angiograms in the DICOM database 202 may correspond to a large set of anonymized patients. A training dataset may be constructed by selecting a subset of CTA scans from the DICOM database 202 to represent a large population (reflecting diversity in data by age and gender) of healthy and unhealthy anonymized patients for training one or more neural network models. A subset of the CTA scans may also be used as a test dataset for testing the efficacy of a neural network model. An Arterial Phase Extraction process 204 may be used to extract the arterial phase and axial view CTA scans in the DICOM database 202. Prior to using the images data for training one or more neural network models, an Image Conversion process 206 may be used to convert CTA scans from DICOM format to a 2-dimensional (2D) Image format (e.g., PNG-portable network graphics image format) for the training and test datasets. However, this is merely an example of how training and test datasets may be formed to train one or more neural networks, and the scope of the claimed subject matter is not limited in this respect.

In one or more embodiments, CTA scans in the DICOM Database 202 may be used by an Arterial Phase Extraction method 204. It is possible that CTA scans in the DICOM Database 202 may include some scans that were taken during the non-contrast phase of the CT scanning process, i.e., before a radiocontrast dye was administered to the patient. After the radiocontrast dye had been injected, postcontrast phases of the CT scanning process may include an arterial phase, venous phase, and delayed phase. In an embodiment, an Image Extraction method 204 may be used to extract all axial view CTA scans out of the CTA scans database 202 that were taken during the arterial phase(s) of the CT scanning process. The Image Extraction method 204 may be configured to use one or more techniques for identifying CTA scans that were taken during the arterial phase. One of these techniques may use a plurality of DICOM's metadata tags for the determination of ‘time’. The determination of ‘time’ interval between the dye administration time and commencement of the CT scanning process validates the CTA scans that are to be used for the development and testing of neural network models. Another metadata tag, for example ‘ContrastBolusStartTime’ tag may indicate the exact moment when a contrast dye was administered to a patient. The ‘ContrastBolusAcquisitionTime’ metadata tag may refer to the specific moment where CTA Scan acquisition commences after the injection of the contrast dye. For arterial phase angiograms, ‘ContrastBolusAcquisitionTime’ may be 15-30 seconds after the ‘ContrastBolusStartTime’ when contrast (dye) was administered. Another validation technique for ensuring that the axial view CTA scans were captured during the arterial phase of the CT scanning process may comprise of examining the ‘ImageComments’ tag, and if true for labels such as ‘CTA’ or ‘Arterial’, and the ‘ImageType’ tag to be true for the label ‘ORIGINAL PRIMARY AXIAL’. However, this is merely an example of how the axial view of a CTA scan is validated and selected during the arterial phase extraction process, and the scope of the claimed subject matter is not limited in this respect.

Image Conversion process 206 in one or more embodiments may further include converting CTA Scan (DICOM) images into 2-dimensional (2D) PNG image format. The Hounsfield Unit (HU) values of the DICOM images are mapped to their respective grayscale pixel values to highlight radiodensities of one or more structures (e.g., tissues, bones, arteries, and organs) in a human body for improving visualization. Bone Windowing protocol (using a minimum value of −500 HU and a maximum value of +1,300 HU) may be used to highlight one or more features including Calcified Plaque and Non-calcified Plaque in an arterial class and/or arteries. HU values may be transformed, using a linear scaling formula, and are mapped to a pixel intensity range of 0 to 255 for an 8-bit PNG image conversion. Mathematical formula for converting an HU value (h) into its corresponding pixel value (p) is as follows:

$$p = ( h - min ) / ( max - min )$$

For example, in case of a Bone Windowing protocol, an HU value of −500 corresponds to a pixel value of 0, representing areas of low density such as fat or air. An HU value of 0 maps to a pixel value of 71, while an HU value of 600 corresponds to a pixel value of 153. Finally, an HU value of +1,300 corresponds to a pixel value of 255, indicating very dense structure, e.g., a bone. Image Conversion Process 206 enables an accurate visual representation of different tissue types as illustrated in FIG. 4, further aiding in the annotation of data with more accuracy prior to using it for training one or more neural network models. However, this is merely an example of how an image conversion process may convert CTA scan (DICOM) images into 2D PNG image format, and the scope of the claimed subject matter is not limited in this respect.

In some embodiments, as illustrated in FIG. 3, images present in the training dataset, after Image Conversion 206, may be communicated to a method 208 for developing a convolutional neural network Recognition Model. As illustrated in FIG. 5, method 208 for developing a Recognition Model, for recognizing multiple regions (of the Peripheral Arterial System) that are present in the images, may comprise: a Data Structuring method 220 and a training method 225. As illustrated in the block diagram 218 in FIG. 6, the Data Structuring Method 220 may create one or more subsets of images (220-A, 220-B, through 220-N), wherein each subset may include images of arterial classes corresponding to only one of the regions of the Peripheral Arterial System. In another embodiment, the training method 225 may train the convolutional neural network Recognition Model 400 to recognize images corresponding to different regions of the Peripheral Arterial System. In another embodiment, test dataset images after their conversion 206, may be used to test the predictions being made by the Recognition Model 400 after each learning phase is over. The structure and parameters of the Recognition model 400 may be further tweaked by iteratively updating its parameters to minimize a defined loss function. However, this is merely an example of how training and test datasets may be structured to train and develop a convolutional neural network Recognition Model, and the scope of the claimed subject matter is not limited in this respect.

As illustrated in FIG. 6, Data Structuring method 220, for training and developing a convolutional neural network Recognition Model 400, may prepare and organize the images dataset of anonymized patients in a plurality of folders (220-A, 220-B, through 220-N), with each folder comprising of images of the arterial classes corresponding to only one region of the Peripheral Arterial System. Different structures (e.g., bones, and soft tissue organs) in a human body may serve as landmarks to assist in marking the start and/or end points of the arterial classes for a region in a series of images. Start and end points of the arterial classes for a region relative to unique landmarks may also be stored in a folder, in addition to storing the images. There are variations in human anatomy; therefore, a certain degree of tolerance may be applied while marking the start and end points of the arterial classes for a region in the images of consecutive slices of a CTA scan for a patient. Additionally, there may be subtle variations in the selection of landmarks to mark the start and end points of different arterial class images in a region. For example, starting point of an arterial class may align with the mid-point of T12 vertebrae (using it as a unique landmark) in a patient, whereas the landmark for the starting point of another patient's arterial class may align with the bottom of T12 vertebrae. In addition to using axial view, coronal and sagittal views may also be used for cross verification purposes during the data structuring process 220. FIG. 7 illustrates the use of all three types of views (i.e., axial, coronal, and sagittal) during the data structuring process to serve as an example of using landmarks to mark the starting image and ending image of a region (e.g., Aortoiliac and Visceral Arterial Region). Application of a managed tolerance does not impact any region recognition or computational processes. However, this is merely an example of how training and test datasets may be structured and used to train a convolutional neural network Recognition Model, and the scope of the claimed subject matter is not limited in this respect.

Embodiments may include a Training method 225 for training a convolutional neural network Recognition Model 400 to learn how to recognize a region's arterial classes that may be present in the training images dataset. During the learning process, the Recognition Model may learn to identify the arterial classes within a region using its unique patterns, textures, and features, including the shapes and unique landmarks associated with said arterial classes. The Training method 225 may deploy a plurality of networked layers of neural network nodes during the learning process. In this method, output of a neural network node configured in a layer may be fed as an input to a neural network node in the next layer. However, increasing the depth of a convolutional neural network, by adding more layers to it, may reduce the accuracy of the resulting model instead of enhancing it. Therefore, a convolutional neural network may also be configured as a residual neural network model to increase its accuracy. In a residual neural network, in addition to feeding the outputs to the nodes in the next layer, the Training method 225 may also relate to one or more methods for feeding the output of a node in a layer to another node which is configured in a different layer while skipping one or more nodes in the intermediate layers. The Training method 225 may improve a neural network's accuracy by optimizing neural network's parameters to minimize the difference between the predicted output and the actual target values in the anonymized training image and test image datasets. An iterative process is used for the training and testing of the convolutional neural network Recognition Model 400. While iterating through the learning process, it may become necessary to add more images or eliminate some of the images from the training and test image datasets for improving performance of the recognition model. However, this is merely an example of how to train a residual convolutional neural network Recognition Model, and the scope of the claimed subject matter is not limited in this respect.

In one or more embodiments, as illustrated in FIG. 3, images present in the training dataset, after Image Conversion 206 may be communicated to a method 210 for developing a plurality of Classification Models. Each Classification Model for a region of the Peripheral Arterial System is trained to categorize and classify the arterial classes and arteries present in said region. As illustrated in FIG. 8, the method 210 for developing a plurality of Classification Models 500 may comprise: a Data Structuring method 235 and a training method 240. For data structuring purposes, images dataset of anonymized patients is saved in a plurality of folders with each folder comprising of images of all body structures including arterial classes in a specified region. FIG. 9 (Abdominal Aorta Arterial Class in Region A), FIG. 10 (Common iliac Arterial Class in Region A), and FIG. 11 (External iliac Arterial Class in Region A) illustrate few examples of training images and data structuring required prior to using them for training a convolutional neural network Classification Model for each region. A folder may be named as Region A, where a subfolder may be named as Abdominal Aorta as its arterial class name as illustrated in FIG. 9, and a subfolder may be named as Common Iliac as its arterial class name as illustrated in FIG. 10, and a subfolder may be named as External Iliac as illustrated in FIG. 11. A similar approach may be used for organizing the folders and subfolders for all other regions as well. Different structures such as bones, soft tissue organs, etc., may serve as unique landmarks to assist in marking the start or end points of an arterial class in a series of images. Information about the start and end points of an arterial class relative to unique landmarks may also be stored in folders in addition to storing the starting point image, end point image, and all images in between the starting point and end point. There are variations in human anatomy; therefore, a certain degree of tolerance may be applied while marking the start and end points of an arterial class in a series of consecutive slices of a CTA scan for a patient. Additionally, there may be subtle variations in the selection of unique landmarks to mark the start and end points of different arterial classes. For example, starting point of an arterial class may align with the mid-point of T12 vertebrae (using it as a unique landmark) in a patient, whereas the unique landmark for the starting point of another patient's arterial class may align with the bottom of T12 vertebrae. In addition to using axial view, coronal and sagittal views may also be used for cross verification purposes during the data structuring process 235. FIG. 7 illustrates the use of all three types of views (i.e., axial, coronal, and sagittal) during the data structuring process to serve as an example of using landmarks to mark the starting image and ending image of a region (e.g., Aortoiliac and Visceral Arterial Region). Application of a managed tolerance does not impact any arterial class' classification or computational processes. However, this is merely an example of how training and test data sets may be structured and used to train and develop a plurality of convolutional neural network Classification Models, and the scope of the claimed subject matter is not limited in this respect.

As illustrated in FIG. 8, embodiments may include a Training method 240 for training a plurality of neural network Classification Models 500 to train each classification model for a region of the Peripheral Arterial System to learn how to categorize each one of the arterial classes present in the images dataset for said region. During the learning process, each classification model may learn to categorize each arterial class image using its unique patterns, textures, and features including the shapes and unique landmarks associated with said arterial class image. For each Classification Model being trained to categorize arterial class images for a region, the Training method 240 may deploy a plurality of networked layers of neural network nodes during the learning process for each of the Classification Models 500 (500-A, 500-B through 500-N). Output of a neural network node configured in a layer of a Classification Model may be fed as an input to a neural network node of said Classification Model in the next layer. However, increasing the depth of a convolutional neural network, by adding more layers to it, may reduce the accuracy of the resulting model instead of enhancing it. Therefore, a convolutional neural network may also be configured as a residual neural network model to increase its accuracy. In a residual neural network, in addition to feeding the outputs to the nodes in the next layer, the Training method 240 may also relate to one or more methods for feeding the output of a node in a layer to another node which is configured in a different layer of a model while skipping one or more nodes in the intermediate layers. The Training method 240 may improve a neural network's accuracy by optimizing each neural network Classification Model's parameters to minimize the difference between the predicted output and the actual target values in the anonymized training image and test image datasets. An iterative process is used for the training and testing of the neural network during the training of Classification Models 500 (500-A, 500-B through 500-N) as illustrated in FIG. 12. While iterating through the learning process, it may become necessary to add more images or eliminating some of the images from the image datasets for improving performance of the classification models. The structure and parameters of the Classification Models 500 may be further tweaked by iteratively updating their parameters to minimize a defined loss function. However, this is merely an example of how a plurality of residual convolutional neural network Classification models may be trained and developed, and the scope of the claimed subject matter is not limited in this respect.

In one or more embodiments, as illustrated in FIG. 3, images present in the training dataset, after Image Conversion 206, may be communicated to a method 212 for the development of a plurality of Segmentation Models. Each Segmentation Model for a region of the Peripheral Arterial System is trained to segment the input images and generate corresponding masks for said region. As illustrated in FIG. 13, method 212 for developing a plurality of Segmentation Models 600 may comprise: a Data Labeling and Mask Generation method 255, a Data Structuring method 260, and a training method 265. The Data Labeling and Mask Generation method 255 may be used for generating a mask for each image in the input image dataset. Input images and their masks may be used for training a plurality of neural network Segmentation Models 600 to learn how to segment input images and create masks of images for each region including images of arterial classes and their arteries (members of an arterial class) in the Peripheral Arterial System. The Data Labeling method 255 may assign specific labels to key features present in each image that may include but not limited to arterial boundary, blood flow, calcified plaque, and non-calcified plaque. Each of the key features may be differentiated and labeled according to the value of their fixed pixel intensity in an input image, e.g., pixel intensity value of 180 for blood flow, 80 for non-calcified plaque, 255 for calcified plaque, and 0 for background. A language-independent file format (e.g., JSON) may be used to store said labels and marked coordinates of features as name-value pairs and arrays. The Labeling and Mask Generation method 255 may store labels and marked coordinates in a separate folder for each region, which may be used to segment each input image and create its mask. FIG. 14 illustrates an example of data labeling and mask generation of an unlabeled image where the boundary of an arterial class and/or artery (member of an arterial class) and a plurality of features within it have been labeled after zooming into it. FIG. 15, illustrates an example of a generated mask of the same image that may be created by using the Labeling and Mask Generation method 255. A mask may be standardized to a dimension of 512×512 pixels for height and width by adjusting an image's pixel array. However, this is merely an example of how image data may be labeled, in addition to segmenting an image to create a mask, prior to using it for training and developing a plurality of convolutional neural network Segmentation Models, and the scope of the claimed subject matter is not limited in this respect.

In one or more embodiments, image data, after Data Labeling and Mask Generation, may be communicated to a Data Structuring method 260 for preparing and organizing image data of anonymized patients and corresponding masks in different folders. All input images of a region of the Peripheral Arterial System are stored in a separate folder as illustrated in FIG. 16 as an example for region A (e.g., Aortoliac and Visceral Arterial Region). Similarly, all masks for each region are stored in a separate folder as illustrated in FIG. 17 as an example for region A (e.g., Aortoliac and Visceral Arterial Region). However, this is merely an example of how images and corresponding masks may be structured, prior to using them for training and developing a plurality of convolutional neural network Segmentation Models, and the scope of the claimed subject matter is not limited in this respect.

As illustrated in FIG. 18, embodiments may include a training method 265 for training a plurality of convolutional neural network Segmentation Models 600 to train a segmentation model, for each region of the Peripheral Arterial System, to learn how to segment each input image in said region for generating a mask for each input image highlighting one or more features in it. The list of features highlighted may include, but not limited to arterial boundary, blood flow, calcified plaque, and non-calcified plaque. For each Segmentation Model being trained for a region, the training method 265 may deploy a plurality of networked layers of neural network nodes during the learning process for each of the Segmentation Models 600 (600-A, 600-B through 600-N). Output of a neural network node configured in a layer of a Segmentation Model may be fed as an input to a neural network node of said Segmentation Model in the next layer. However, increasing the depth of a convolutional neural network, by adding more layers to it, may reduce the accuracy of the resulting model instead of enhancing it. Therefore, a convolutional neural network may also be configured as a residual neural network model to increase its accuracy. In a residual neural network, in addition to feeding the outputs to the nodes in the next layer, the Training method 265 may also relate to one or more methods for feeding the output of a node in a layer to another node which is configured in a different layer of a model while skipping one or more nodes in the intermediate layers. The Training method 265 may improve a neural network's accuracy by optimizing each neural network Segmentation Model's parameters to minimize the difference between the predicted output and the actual target values in the anonymized training image and test image datasets. An iterative process is used for the training and testing of the neural network during the training of Segmentation Models 600 (600-A, 600-B through 600-N) as illustrated in FIG. 18. While iterating through the learning process, it may become necessary to add more images or eliminating some of the images from the image datasets for improving performance of the segmentation models. The structure and parameters of the Segmentation Models 600 may be further tweaked by iteratively updating their parameters to minimize a defined loss function. However, this is merely an example of how a plurality of residual convolutional neural network Segmentation Models may be trained and developed, and the scope of the claimed subject matter is not limited in this respect.

In one or more embodiments, as illustrated in FIG. 3, image data in the training dataset, after Image Conversion 206, may be communicated to a method 214 for the development of a plurality of Artery Labeling Models where each model corresponds to a specific arterial region. As illustrated in FIG. 19, method 214 for developing a plurality of Artery Labeling Models 755 may comprise: a Data Labeling and Mask Generation method 255, a Bitwise AND Operation method 275, a Data Structuring method 280, and a training method 285. The Data Labeling and Mask Generation method 255 may be used for generating a mask and label(s) for each artery present in the input image dataset. The label(s) for each image in the input image dataset may be stored in the language-independent file format (e.g., JSON) of respective image and/or data dictionary, in which each resultant arterial image may be mapped to the corresponding label name and its respective bounding box coordinates. However, this is merely an example of how image dataset may be labeled and the masks may be generated, and the scope of the claimed subject matter is not limited in this respect.

In some embodiments, input images and their corresponding masks may be communicated to the Bitwise AND Operation method 275 on each input image and its corresponding mask (as illustrated by an example in FIG. 20) to generate a resultant arterial image (as illustrated by an example in FIG. 21). The resultant arterial image(s) and their corresponding label(s) may be communicated to a Data Structuring for Artery Labeling Models method 280, preparing and organizing resultant arterial images and label(s) data of anonymized patients in a folder, prior to training a plurality of Artery Labeling Models as illustrated in FIG. 19. However, this is merely an example of how a Resultant Arterial Image may be transformed, structured, and added to a dataset for training a plurality of neural network Artery Labeling Models, and the scope of the claimed subject matter is not limited in this respect.

Embodiments may include a Training method 285 for training a plurality of neural network Artery Labeling Models 755 (755-A, 755-B through 755-N) as illustrated in FIG. 22, wherein each artery labeling model may receive the resultant arterial image, one at a time, along with the label(s), as part of a batch of resultant arterial images. Model training parameters, such as weights, may be fine-tuned for each epoch until the results of the models meet each model's performance goals. The performance goals may be achieved upon satisfactory and/or accurate prediction of bounding boxes and/or location coordinates of different arteries (for example, Hepatic artery, Splenic artery, and Renal arteries of an arterial class for Abdominal Aorta). During the learning process, each artery labeling model may learn to label different arteries present in a resultant arterial image based on their unique features, e.g., shape, location, and contrast. In the training method 285, an artery labeling model may learn to process each input resultant arterial image by extracting feature maps using a convolutional neural network configured with multiple layers. Output of a neural network node configured in a layer of an Artery Labeling Model may be fed as an input to a neural network node of said Artery Labeling Model in the next layer. However, increasing the depth of a convolutional neural network, by adding more layers to it, may reduce the accuracy of the resulting model instead of enhancing it. Therefore, a convolutional neural network may also be configured as a residual neural network model to increase its accuracy. In a residual neural network, in addition to feeding the outputs to the nodes in the next layer, the Training method 285 may also relate to one or more methods for feeding the output of a node in a layer to another node which is configured in a different layer of a model while skipping one or more nodes in the intermediate layers. A residual neural network model may be configured as a Faster R-CNN with a Feature Pyramid Network (FPN). Additionally, this residual neural network may also include a region (of interest) proposal network (RPN), a region of interest (ROI) pooling layer, an object detection and classification layer, and a bounding box regression head. The Training method 285 may improve a neural network's accuracy by optimizing each neural network Artery Labeling Model's parameters to minimize the difference between the predicted output and the actual target values. An iterative process is used for the training and testing of the neural network during the training of Artery Labeling Models 755 (755-A, 755-B through 755-N) as illustrated in FIG. 22. While iterating through the learning process, it may become necessary to add more resultant arterial images or eliminating some of the images from the resultant arterial image datasets for improving performance of the Artery Labeling models. The structure and parameters of the Artery Labeling Models 755 may be further tweaked by iteratively updating their parameters to minimize a defined loss function. In addition to extracting feature representations, the training method may relate to one or more methods for learning to predict artery label(s) within a resultant arterial image by integrating an internal region of interest (ROI) proposal mechanism, reducing computational overhead and improving accuracy. However, this is merely an example of how a plurality of residual convolutional neural network artery labeling models may be trained and developed, and the scope of the claimed subject matter is not limited in this respect.

Referring now to FIG. 23 illustrating a preferred use case embodiment 300 of pre-trained neural networks to analyze medical images of a known patient, diagnose possible disease of said patient, conduct risk analysis, facilitate disease tracking, and assist in clinical decision-making. In one or more embodiments, CTA scans or angiograms of said known patient may be captured with the help of a medical image acquisition device 105, stored in a Picture Archiving and Communication Server (PACS) database 306 along with patient's identifying information, and retrieved, automatically and/or on demand, from the PACS database. Known patient's identifying information is entered using an input method 304. Patient's identifying information may include patient's name, medical record number (MRN), date of birth, gender, current address, and any other information required for identification and clinical purposes. PACS database may be locally accessible by the system and/or may be accessed remotely through a network. Medical images may be stored in the form of a single (pre-processed) and/or multiple (post-processed) DICOM-formatted CTA scans of one or more peripheral arterial regions of the known patient. However, this is merely an example of how CTA scans of a known patient may be stored in a medical images database, and the scope of the claimed subject matter is not limited in this respect.

In the current embodiment, the disclosed system may be initiated with a request for retrieving known patient's CTA scans for performing a clinical diagnosis. Request for retrieving known patient's CTA scans and diagnosis may be triggered by a clinician or generated programmatically 320. A clinician may use a proprietary user interface to retrieve known patient's CTA scans for diagnosis purposes by entering said patient's identifying information (medical record number (MRN) and/or patient's name). A clinician may be a lab technician, medical lab staff, vascular specialist, and/or a radiologist. The clinician may be presented with multiple records of CTA scans which may have been captured during a single CT scanning session or in multiple scanning sessions over a period of time. Each CT scanning session may capture CTA scans for one or more regions of known patient's peripheral arterial system. Clinician may select appropriate records for processing by the system. Similarly, the request for retrieving known patient's CTA scans for performing a clinical diagnosis may be generated programmatically and appropriate records selected for processing by the system. System may retrieve selected CTA scans or angiograms from the database 306 and communicate to the arterial phase extraction process 204. In an embodiment, arterial phase extraction process 204 may extract all axial view CTA scans out of the CTA scans database 306 that were taken during the arterial phase(s) of the CT scanning process. The arterial phase extraction process 204 may be configured to use one or more techniques (including the use of metadata tags) for identifying CTA scans that were taken during the arterial phase. However, this is merely an example of how the system may receive a request for performing a clinical diagnosis for a known patient, retrieve patient's CTA scans, extract axial view images, and the scope of the claimed subject matter is not limited in this respect.

Angiograms data (in DICOM), after arterial phase extraction, may comprise pixel array information and DICOM tags, e.g., Image Position Patient (IPP), image Instance Number (IN), Slice Interval (SI). In one or more embodiments, as illustrated in FIG. 23, an Image Conversion method 206 may be configured to convert CTA scans or angiograms (in DICOM) of a known patient into 2D PNG images files 325, and DICOM Tags files 245. Each PNG images file may be saved, using a file naming convention, in a file system or in a database. The file naming convention may include known patient's id (PID) and DICOM's image's instance number for each PNG image file. Similarly, each DICOM tags file may be saved in a tabular file format, e.g., comma separated values (CSV) format in a database/filesystem, and it may comprise a plurality of columns including a column for each extracted tag value. Additionally, it may include PNG image filenames corresponding to each column with a tag value. However, this is merely an example of how an image conversion process may convert and save angiogram data into 2-dimensional (2D) PNG image files and DICOM tags files, and the scope of the claimed subject matter is not limited in this respect.

In some embodiments, a computer-implemented method 400, which may comprise a pre-trained residual neural network Recognition Model, is configured to access 2D PNG images from a filesystem/database 325, and to recognize each image to belong to one of the regions of the peripheral arterial system. The residual neural network Recognition Model 400 may predict and recognize a region of the peripheral arterial system on the basis of its unique features and associated landmarks. List of the regions of peripheral arterial system may comprise Brachioaxillary Arterial Region, the Radioulnar Arterial Region, the Aortoiliac and Visceral Arterial Region, the Femoropopliteal Region, and the Crural Region. All 2D PNG images that correspond to a recognized region of the peripheral arterial system might be saved in a regional images database 425 under a unique folder (or a digital container) for said region. However, this is merely an example of how 2D PNG images of a region of the peripheral arterial system may be recognized and, separated, and saved in a unique folders for said region by using a pre-trained residual neural network Recognition Model, and the scope of the claimed subject matter is not limited in this respect.

In one or more embodiments of the present invention, number of regions (nr) of the peripheral arterial system to be diagnosed may be deduced by the computer-implemented method 426 from the number of regions that may have been recognized, and saved in the regional images database 425 by the Recognition Model 400. Alternatively, number of regions (nr) to be diagnosed may be entered with the help of said computer-implemented method 426. However, this is merely an example of how the number of regions of the peripheral arterial system is determined for diagnostic purposes, and the scope of the claimed subject matter is not limited in this respect.

In one or more embodiments of the present invention, as illustrated in FIG. 23, the system is configured to iterate over one or more regions of the peripheral arterial system for diagnosing disease. Prior to starting the iterative process, a computer-implemented method 428 may assign a value of 1 to a variable i representing current-region of the peripheral arterial system being processed. Each iteration starts at connector block 430. During each iteration, method 432 processes all PNG images belonging to the current-region i of the peripheral arterial system. Processing Block 432 may include multiple sub-processes, which are described in subsequent embodiments. Before processing the images for the next region of the peripheral arterial system, the system is configured to increment the current-region variable i by 1 in step 496. Following this increment, at conditional block 498, the system may determine whether the current-region variable i exceeds the threshold value nr representing total number of regions to be processed before proceeding further. If the current-region variable i≤nr, next iteration may be initiated from the connector block 430 with the incremented value of i. Otherwise, if i>nr, the system may terminate the loop. However, this is merely an example of how the system is configured to process PNG images of one or more regions of the peripheral arterial system for diagnosing disease, and the scope of the claimed subject matter is not limited in this respect.

In one or more embodiments, as illustrated in FIG. 24, a flowchart 405 illustrates sub-processes of the processing Block 432, detailing computer-implemented methods for utilizing a pre-trained residual neural network Classification Model for the current-region i to categorize arterial classes in it, and a pre-trained residual neural network Segmentation Model for segmenting the images for the current-region i. The process may begin at Block 436, where the threshold value for the total number of images, associated with the peripheral arterial region i, is retrieved from a database and/or file system, referred to as Region(s) Images 425, which are then processed sequentially. In one or more embodiments, as illustrated in FIG. 24, the system is configured to iterate over one or more images of the current-region i of the peripheral arterial system for processing. Prior to starting the iterative process, a computer-implemented method 438 may assign a value of 1 to a variable j representing current-image number of the current-region i being processed. Each iteration starts at connector block 440 for processing the current-image number j of the current-region i. The PNG image corresponding to the current-image number j for the current-region i is retrieved from the Region(s) Images database 425 in step 442 for further processing as described in subsequent embodiments. Before processing the next image, the system is configured to increment the current-image number variable j by 1 in step 444. Following this increment, at conditional block 446, the system may determine whether the current-image number variable j exceeds the threshold value for total number of images to be processed before proceeding further. If the current-image number variable j is less than the threshold value for the total number of images, next iteration may be initiated from the connector block 440 with the incremented value of j. Otherwise, if j is greater than the threshold value for the total number of images, the system may terminate the loop. However, this is merely an example of how a pre-trained residual neural network Classification and Segmentation models may be used for processing images for a region of the peripheral arterial system, and the scope of the claimed subject matter is not limited in this respect.

In one or more embodiments, after retrieving the PNG image corresponding to the current-image number j for the current-region i from the database 425 in step 442, said image may be fed to a pre-trained residual neural network Classification Model for the region i at Block 500. The Classification Model may subsequently predict the arterial class present in the image current-image number j. The disclosed system is configured to transmit the filename of the current-image number j, along with its corresponding arterial class, to the Processing Block 510. Processing Block 510 may store the filename of said image along with its predicted class in a comma-separated values (CSV) file, designated as regions' CSV, in Database 620. The CSV file may comprise of two columns, one for the image filename and the other for the predicted class of the image. However, this is merely an example of how pre-trained residual neural network Classification Models may categorize and classify the results and how those predicted results may be saved, and the scope of the claimed subject matter is not limited in this respect.

In some embodiments, at Block 600, the disclosed system is configured to generate a predicted mask for the retrieved current-image number j in the current-region i by feeding said image to a residual neural network Segmentation Model 600. The predicted mask may have specific labels for each key features present in the current-image number j that may include but are not limited to arterial boundary, blood flow, calcified plaque, non-calcified plaque, and/or aneurysm. Each key feature in the predicted mask may be differentiated according to the prescribed values of their respective grayscale pixel intensities, e.g., pixel intensity value of 180 for blood flow, 80 for non-calcified plaque, 255 for calcified plaque, and 0 for background, as previously described during model training. Subsequently, the current-image number j, along with its corresponding predicted mask, may be forwarded to Processing Block 610. At Processing Block 610, the disclosed system is configured to store the predicted mask in a Database and/or File System 640, referenced to as Region(s) Masks. The stored mask may be organized in a structured format (e.g., with the same index as the input image, and saved in a folder to facilitate subsequent retrieval and further processing. However, this is merely an example of how a pre-trained residual neural network Segmentation Model for a region of the peripheral arterial system may be used and how its predicted results may be saved, and the scope of the claimed subject matter is not limited in this respect.

In one or more embodiments, after processing all images using residual neural network classification and segmentation models for the current-region i, execution may proceed, as illustrated in FIG. 25, to a noise filtering process for the arterial classes which have been predicted by the residual neural network classification model for the current-region i. The flowchart diagram 410 illustrates one or more embodiments of a method where a noise filtering process may be applied to the classification results for the arterial classes that were saved in the Database 620 for the current-region i. The disclosed system is configured to remove inconsistencies and/or noise from the classification predictions made in the regions' CSV file stored in the Database 620. Some inconsistencies, or noise, may occur when an image within a sequence of classified images has been misclassified, deviating from the actual target class. The process may begin at Block 312, where the threshold value for the total number of predicted arterial classes, associated with the peripheral arterial region i, is retrieved from a database and/or file system, referred to as Region(s) CSV 620. Predicted arterial classes are then processed sequentially. In one or more embodiments, as illustrated in FIG. 25, the system is configured to iterate over one or more predicted arterial classes of the current-region i of the peripheral arterial system for processing. Prior to starting the iterative process, a computer-implemented method 448 may assign a value of 1 to a variable k representing current-arterial-class number in the current-region i being processed. Each iteration starts at connector block 449 for processing the current-arterial-class number k in the current-region i. Current arterial class k for the current-region i is retrieved from the Region's CSV database 620 in steps 451. System is configured to remove inconsistencies and/or noise from the predictions made for the current arterial class k as described in subsequent embodiments. Before processing the next predicted arterial class, the system is configured to increment the current-arterial-class number variable k by 1 in step 458. Following this increment, at conditional block 461, the system may determine whether the current-arterial-class number variable k exceeds the threshold value for total number of predicted arterial classes to be processed before proceeding further. If the current-arterial-class number variable k is less than the threshold value for the total number of predicted arterial classes, next iteration may be initiated from the connector block 449 with the incremented value of k. Otherwise, if k is greater than the threshold value for the total number of predicted arterial classes, the system may terminate the loop. For example, as shown in FIG. 26, a sequence of images, along with their predicted classes, has been classified by a residual neural network classification model for region i of the peripheral arterial system. In this example, within a sequence of image files for an arterial class (e.g., abdominal aorta), an anomaly has been detected in image9.png (e.g., external iliac), where the predicted class differs from the arterial class predicted for the majority of the images in the image sequence, and may be considered as an inconsistency, an outlier, and/or noise. The disclosed noise-filtering process is designed to correct similar prediction errors by ensuring uniformity in predictions across the sequence of arterial class images. However, this is merely an example of how inconsistencies and/or noise may be removed from the classification predictions made for a sequence of images for a region of the peripheral arterial system, and the scope of the claimed subject matter is not limited in this respect.

In some embodiments, the system may be configured to iterate over the arterial classes associated with the region i of the peripheral arterial system as illustrated in FIG. 25. At Processing Block 451, the system may retrieve unique arterial class data for the current-arterial-class k from the regions' CSV that had been stored within the Database and/or File System 620. The retrieved in a CSV file may comprise of two columns, one containing image file names and the other containing corresponding predicted arterial class name, classified by the Classification Model for region i of the peripheral arterial system. At processing Block 453, the system may analyze the retrieved arterial class column to identify any inconsistencies, outlier(s) and/or noise, for corresponding classified image(s). For example, to correct a misclassification within an arterial class sequence of images, the system may rename the misclassified class for a misclassified image as illustrated in FIG. 26. At Processing Block 455, the filtered data may either be modified where noise is detected or left as it is in the case of undetected noise. The updated values may be stored in a structured table format, e.g a comma-separated values (CSV) file, with one column representing image file names and the other column containing the filtered predicted classes names, referred to as Modified Region's CSV in a Database and/or File System 456. However, this is merely an example of how Noise Filtering methods may be applied to the data of predicted arterial classes for a region of the peripheral arterial system, and the scope of the claimed subject is not limited in this respect.

Referring to FIG. 27, Diagram 415 is a flowchart illustrating an overview of example embodiments of a method that may apply a Noise Filtering method to the image masks which were predicted by the residual neural network Segmentation Model for the current-region i of the peripheral arterial system. The disclosed system may be configured to remove noise from image masks which were previously stored in a region's masks Database and or in a File System 640. Some noise in an image mask may consist of isolated pixel clusters and/or unwanted areas within a predicted mask. The process may begin at Block 314, where the threshold value for the number of the last image mask, associated with the peripheral arterial region i, is retrieved from a database and/or file system, referred to as Region(s) Masks 640. Predicted image masks are then processed sequentially. In one or more embodiments, as illustrated in FIG. 27, the system is configured to iterate over one or more predicted image masks of the current-region i of the peripheral arterial system for processing. Prior to starting the iterative process, a computer-implemented method 464 may assign a value of 1 to a variable s representing current-image-mask number in the current-region i being processed. Each iteration starts at connector block 466 for processing the current-image-mask number s in the current-region i. Current current-image-mask number s and the next mask (numbered s+1) for the current-region i are retrieved from the Region's Masks database 640 in steps 468. System is configured to remove inconsistencies and/or noise from the current-image-mask number s as described in subsequent embodiments. Before processing the next image mask, the system is configured to increment the current-image-mask number s by 1 in step 474. Following this increment, at conditional block 476, the system may compare the current-image-mask number variable s with the threshold value for the number of the last image mask to be processed before proceeding further. If the current-image-mask number variable s is not equal to the threshold value for the number of the last image mask, next iteration may be initiated from the connector block 466 with the incremented value of s. Otherwise, if s is equal to the threshold value for the number of the last image mask, the system may terminate the loop. As illustrated in FIG. 28, an image mask that was predicted by a segmentation model for a region of the peripheral arterial system is exhibited as input. In this example, an anomaly or noise is present in the predicted mask. The disclosed Noise Filtering method 415 is designed to correct prediction errors made by a Segmentation Model to ensure uniformity in predictions across the image masks. The input image mask, after isolated pixel cluster(s) and/or unwanted area(s) have been removed by applying the Noise Filtering method 415 is exhibited as output in FIG. 28. However, this is merely an example of how isolated pixel cluster(s) and/or unwanted area(s) may be removed from the image masks for a region of the peripheral arterial system, and the scope of the claimed subject matter is not limited in this respect.

In some embodiments, the system may be configured to iterate over the image masks associated with the region i of the peripheral arterial system as illustrated in FIG. 27. In some embodiments, at Processing Block 468, the system may retrieve current-image-mask number s and the next mask (numbered s+1) from a sequence of masks for the current-region i from the Database and/or the File System 640. The image mask numbered s may have one or more areas of interest (AOIs), i.e., arterial classes and/or arteries, in it. Both masks numbered s and s+1 may contain segmented areas of interest, and/or noise in them where respective sets of noise contours in them may be referred to as contour C₁ for image mask s and contour C₂ for image mask s+1. In addition to the true arterial class and/or artery or Area of Interest (AOI), noise may be present in both masks (numbered s and s+1). Computation and detection of a noise contour Cₙₒᵢₛₑ for image mask s, in set-theoretic terms, is as follows:

$${C}_{noise} = ( {C}_{1} OR {C}_{2} ) - ( {C}_{1} AND {C}_{2} )$$

Processing Block 470 may process the retrieved current-image-mask number s for noise filtering by assessing the continuity of the segmented arterial class and/or artery in the subsequent image mask s+1. In case of any noise detected in the current-image-mask numbered s, the system may remove its noise based on the computed Cₙₒᵢₛₑ from image mask s. Processing Block 471 may use the Modified Region's Masks database/filesystem 472, to save the modified image mask of the current-image-mask number s after removing the noise (if noise was detected), otherwise save the image mask s as it is (if no noise was present in it). An example of input and output of the noise filtering method is illustrated in FIG. 28. However, this is merely an example of how the Noise Filtering Method may be applied to detect and remove noise from image masks of a region of peripheral arterial system, and the scope of the claimed subject is not limited in this respect.

Referring to FIG. 29, diagram 805 is a flowchart that illustrates an overview of example embodiments of a method used for the labeling of each artery that may be present in one or more PNG images of the current-region i of the peripheral arterial system. The primary objective of the disclosed process may assign label(s) and their respective bounding box coordinates using an Artery Labeling model for the images for the current-region i. The Artery Labeling model is a type of a pre-trained residual neural network model, which is commonly referred to as a Faster R-CNN with a Feature Pyramid Network (FPN). A Bitwise AND Operation method may generate a resultant arterial image, which may be used as an input to an Artery Labeling model previously trained for region i of the peripheral arterial system. The model's output may generate label(s) and their respective bounding box coordinates for one or more arteries present in the PNG images for region i of the peripheral arterial system. The process may begin at Block 720, where the threshold value for the total number of image files, associated with the peripheral arterial region i, may be retrieved from a Modified Region's CSV database/filesystem 456. PNG images, image masks, and their resultant arterial images are then processed in one or more embodiments as illustrated in FIG. 29. Prior to starting an iterative process, a computer-implemented method 722 may assign a value of 1 to a variable u representing a current-counter number to be used while processing each PNG image, its image mask, and the resultant arterial image, for the current-region i being processed. Each iteration starts at connector block 725 for processing a PNG image, its image mask, and a resultant arterial image in the current-region i as described in subsequent embodiments. Before processing the next PNG image, its image mask, and a resultant arterial image, system is configured to increment the current-counter number u by 1 in step 765. Following this increment, at conditional block 768, the system may compare the current-counter variable u with the threshold value for the total number of image files, associated with the peripheral arterial region i before proceeding further. If the current-counter variable u is less than or equal to the threshold value for the total number of image files, next iteration may be initiated from the connector block 725 with the incremented value of u. Otherwise, if u is greater than the threshold value for the total number of image files, the system may terminate the loop. However, this is merely an example of how each artery may be labeled in all PNG images for a region of the peripheral arterial system, and the scope of the claimed subject matter is not limited in this respect.

In some embodiments, the system may be configured to iterate over the PNG images, their image masks, and resultant arterial images associated with the region i of the peripheral arterial system using current-counter u, as illustrated in FIG. 29. In some embodiments, at Processing Block 730, the system may access Modified Region's CSV database/filesystem 456, and retrieve the name of the PNG image file that corresponds to the current-counter u in region i of the peripheral arterial system. At Processing Block 735, the system may retrieve the image mask, corresponding to said PNG image file, from the Modified Region's Masks database/filesystem 472. At Processing Block 740, system may retrieve the 2-dimensional (2D) PNG image from the database/filesystem 325. A Bitwise AND Operation method 275 may be applied to the retrieved PNG image and its corresponding image mask to generate a resultant PNG image and save it in a Resultant Arterial Image database/filename 750. Each bit of each pixel of the resultant arterial image has a non-zero value only and only if the corresponding bits in the retrieved PNG image and its image mask are non-zero. Said resultant arterial image may be fed to a pre-trained Arterial Labeling model for region i. In one or more embodiments, at processing block 755, said Artery Labeling model may render predictions on the resultant arterial image retrieved from the database/filesystem 750 corresponding to the current-counter u. Predictions made by the Artery Labeling model for region i may comprise artery labels and their respective bounding box coordinates. A method 757 may be used to save these predictions in a Labeled Images (CSV) database/filesystem 760. These predictions may be saved in a table structured file format (e.g., a comma separated values CSV file format). However this is merely an example of how an Artery Labeling model may be used to label each artery in an image, generate a bounding box for it, and save each labeled image in a database/filesystem, and the scope of the claimed subject is not limited in this respect.

Referring to FIG. 30, diagram 420 is a flowchart that illustrates one or more example embodiments of the methods that may be used for deploying an Anekanta algorithm. The primary objective of the disclosed Anekanta algorithm may comprise integration of partial predictions that may have been generated, for a region i of the peripheral arterial system, by a plurality of neural network models (e.g., Recognition Model, Classification model, Segmentation model, and the Artery Labeling model). The Anekanta algorithm may be configured to use a set of rules to consolidate data from one or more tabular files into a single unified tabular file format (e.g., CSV). A tabular file may be structured and saved in any database system and/or in a file system in any format that may be suitable for processing, e.g., a comma separated values (CSV) file format. The process may begin at Block 412, where the threshold value for the total number of image files, associated with the peripheral arterial region i, may be retrieved from a Modified Region's CSV database/filesystem 456. Prior to starting an iterative process, a computer-implemented method 478 may assign a value of 1 to a variable m representing a current-file-counter number to be used while processing each PNG image, and the resultant arterial image, for the current-region i being processed. Each iteration starts at connector block 480 for retrieving a PNG image filename, labeled image filename, and image tags in the current-region i prior to applying the Anekanta Algorithm as described in subsequent embodiments. Before processing the next PNG image, labeled image, and image tags, system is configured to apply the Anekanta Algorithm and increment the current-file-counter number m by 1 in step 710. Following this increment, at conditional block 715, the system may compare the current-file-counter variable m with the threshold value for the total number of image files, associated with the peripheral arterial region i before proceeding further. If the current-file-counter variable m is less than or equal to the threshold value for the total number of image files, next iteration may be initiated from the connector block 480 with the incremented value of m. Otherwise, if m is greater than the threshold value for the total number of image files, the system may terminate the loop. Anekanta algorithm may save its results in an Anekanta Results CSV database/filesystem. However, this is merely an example of how an Anekanta algorithm may integrate partial predictions that may have been generated, for a region i of the peripheral arterial system, by a plurality of neural network models, and the scope of the claimed subject is not limited in this respect.

In some embodiments, the system may be configured to iterate over the predictions made by a plurality of residual neural networks for each PNG image associated with the region i of the peripheral arterial system using current-file-counter m, as illustrated in FIG. 30. At Processing Block 482, the configured system may access Modified Region's CSV database/filesystem 456 to retrieve prediction data which may include PNG image's filename, along with the image's arterial class name that had been previously classified by a residual neural network Classification Model for region i, and processed by the noise filtering method for noise removal from the arterial classes of a region. Similarly, at Processing Block 486, the system may be configured to retrieve the labeled arteries prediction data along from labeled image (CSV) database and/or the filesystem 760. At Processing Block 487, the configured system may retrieve the tags information corresponding the PNG image using current-file-counter m file the Tags CSV 245 database and/or filesystem. As discussed herein, the partial predictions data for each image file corresponding to the current-file-counter m may be communicated to an Anekanta algorithm at Processing Block 700. The Anekanta algorithm may combine and/or group all retrieved predictions for said PNG image file corresponding to the current-file-counter m. A computer-implemented method 703 may save the Anekanta algorithm's output in a tabular file format (i.e., CSV file), as referred to Anekanta Result CSV database and/or the filesystem 705. Additionally, this consolidated information may be used to overlay predictions on top of said input PNG image to enable a clinician to visualize the integrated predictions in addition to said PNG image on a display device. However, this is merely an example of how the Anekanta algorithm may be used to integrate predictions made by a plurality of residual neural networks, save consolidated results, overlay consolidated results on top of the input images for visualizion by a clinician, and the scope of the claimed subject matter is not limited in this respect.

Referring to FIG. 31, diagram 435 is a flowchart illustrating an overview and example embodiments of an Inference Engine 800 and a report generation engine 900 for predicting potential disease in the arterial classes of a region i of the peripheral arterial system of a known patient and reporting a diagnosis. The process may begin at Block 312, where the threshold value for the total number of arterial classes, associated with the peripheral arterial region i, may be retrieved from an Anekanta Results CSV database/filesystem 705. Prior to starting an iterative process, a computer-implemented method 488 may assign a value of 1 to a variable n representing a current-arterial-class number to be used while processing each arterial class, for the current-region i being processed. Each iteration starts at connector block 490 for diagnosing potential peripheral arterial disease in the current-arterial-class in region i using a computer-implemented Inference Engine method 800 and reporting the results of the diagnosis using a computer-implemented Report Generation Engine method 900. Refer to subsequent embodiments for a detailed description of the embodiments for both the Inference Engine 800 and Report Generation Engine 900. Before processing the next arterial class for region i, system is configured to increment the current-arterial-class number n by 1 in step 492. Following this increment, at conditional block 494, the system may compare the current-arterial-class variable n with the threshold value for the total number of arterial classes, associated with the peripheral arterial region i before proceeding further. If the current-arterial-class variable n is less than or equal to the threshold value for the total number of arterial classes for region i, next iteration may be initiated from the connector block 490 with the incremented value of n. Otherwise, if n is greater than the threshold value for the total number of arterial classes for region i, the system may terminate the loop. However, this is merely an example of how the system may infer and report potential peripheral arterial disease in one or more arterial classes of a known patient, and the scope of the claimed subject is not limited in this respect.

Referring to FIG. 32, diagram 815 is a flowchart illustrating an overview and example embodiments of an Inference Engine 800 for predicting potential disease in an arterial class of a region i of the peripheral arterial system of a known patient. Prior to executing block 802, a threshold value for the total number of image files for an arterial class n, associated with the peripheral arterial region i, may be determined by a computer-implemented method. Prior to starting an iterative process, a computer-implemented method 802 may assign a value of 1 to a variable q representing a current-image-file number to be used while processing each labeled image file and its mask in the current-arterial-class n, for the current-region i being processed. Each iteration starts at connector block 804 for diagnosing potential peripheral arterial disease in an image mask corresponding to the current-image-file number q in the current-arterial-class n in region i. At processing block 806, the disclosed system may be configured to retrieve the image file name along with its label from the Anekanta Result CSV file from the database/filesystem 705. Similarly, at Processing Block 808, the system may retrieve the corresponding image mask from modified region's masks that may have been previously saved in a database/filesystem 472. System is configured to examine the presence or absence of one or more anomalies in said image mask using a computer-implemented Anomaly Detection method 820 for diagnosing the disease. The Anomaly Detection method 820 may apply a set of rules to detect one or more anomalies based on the differences in pixel intensities. In one or more embodiments as discussed herein, Processing Block 820 may detect anomalous grayscale pixel intensities and/or contrasts other than blood flow in an image mask which may be used to infer the existence of non-calcified plaque, calcified plaque and/or another type of anomaly, and may mark the image mask as suspicious. For example, as illustrated in FIG. 33, image a of an image mask may be described to be normal in which only one grayscale pixel intensity and/or contrast for blood flow is visible in the image mask. Conversely, image b for a mask exhibits multiple grayscale pixel intensities and/or contrasts, marking the mask as suspicious as a result of a possible disease, e.g., calcified plaque, or non-calcified plaque, etc. In some embodiments, if no anomaly is detected in said image mask at condition block 825, the Processing Block 827 may save a null dictionary of volumetric calculation against the retrieved mask in a database/filesystem 875 in an open standard file format (e.g., JSON and/or a dictionary). If one or more anomalies are detected in said image mask corresponding to the current-image-file number q, a computer-implemented may perform volumetric calculations 832 for all anomalies detected in said image mask. Refer to subsequent embodiments for a detailed description of the embodiments of the methods used for performing volumetric calculations. Before processing the next image mask in the current-arterial-class n, for the current-region i being processed, system is configured to increment the current-image-file number q by 1 in step 885. Following this increment, at conditional block 890, the system may compare the current-image-file number q with the threshold value for the total number of image files for an arterial class n, associated with the peripheral arterial region i before proceeding further. If the current-image-file number q is less than or equal to the threshold value for the total number of image files for an arterial class n, associated with the peripheral arterial region i, next iteration may be initiated from the connector block 804 with the incremented value of q. Otherwise, if q is greater than the threshold value for the total number of image files for an arterial class n, associated with the peripheral arterial region i, the system may terminate the loop. However, this is merely an example of how the system may infer peripheral arterial disease in an image mask in an arterial class within a region of the peripheral arterial system of a known patient, and the scope of the claimed subject is not limited in this respect.

Referring to FIG. 34, diagram 818 is a flowchart illustrating an overview of example embodiments for differentiating one or more contours within an image mask and determining total number of unique contours in said image mask. An example of multiple contours, as depicted in FIG. 36, illustrates multiple sub-groups in an image mask wherein each sub-group may be identified as a result of having a different grayscale pixel intensity than other sub-groups. A set of computer-implemented rules may be used to assign a unique semantic value to each area with a uniform grayscale pixel intensity. Different grayscale pixel intensities may represent non-calcified plaque, calcified plaque, blood flow, and arterial boundaries within an image mask. Each of these sub-groups may be identified as a distinct contour with unique grayscale pixel intensity values. By separating and analyzing these contours, the system may accurately differentiate the anatomical structures to perform volumetric calculations. The disclosed system in FIG. 34, diagram 818, as discussed herein, is a sub-process of Processing Block 832. At Processing Block 834, the system may retrieve the image mask numbered q for an arterial class numbered n within the arterial region numbered i from the Modified Region's Masks database/filesystem 472. In one or more embodiments, at Processing Block 836, the system may determine and differentiate unique contour boundaries by analyzing distinct grayscale pixel intensity values within each contour's boundary. This process may facilitate a precise volumetric calculation for each individual contour. At Processing Block 838, the system may save the pixel coordinates marking the boundary of each differentiated contour in the Anekanta Result CSV database/filesystem 705. At Processing Block 842, the system may retrieve a threshold value for the total number of unique contours saved in the Anekanta Result CSV database/filesystem 705. Prior to starting an iterative process for performing volumetric calculations for each contour, a computer-implemented method 844 may assign a value of 1 to a variable r representing a current-contour number to be used while processing each contour in the current-arterial-class n, for the current-region i being processed. However, this is merely an example of how one or more contours may be differentiated in an image mask prior to initiating a volumetric calculations loop for said contours, and the scope of the claimed subject matter is not limited in this respect.

Referring to FIG. 35, diagram 868 illustrates an overview of the embodiments describing a computer-implemented method that may iterate over one or more contours within an image mask for the current-image-file number q, in the current-arterial-class n, for the current-region i of the peripheral arterial system being processed. A computer-implemented method may perform volumetric calculations for the current-contour numbered r where variable r represents the current contour number in an image mask being processed. Each iteration starts at connector block 850 for performing volumetric calculations for the current-contour numbered r. At Processing Block 855, the disclosed system may retrieve the data for the current-contour numbered r from the Anekanta Result CSV database/filesystem 705. In one or more embodiments, at Processing Block 852, the system may perform volumetric calculations on the retrieved current-contour numbered r of image mask q. The methods for making volumetric calculations may comprise multiple calculations including computing the diameter of a contour, radius of a contour, and area of a contour. As illustrated in FIG. 37, an example contour may represent an aneurysm in the abdominal aorta, which may be detected by performing volumetric calculations on said contour. As discussed herein, aneurysm is a permanent localized dilatation of a vessel diagnosed using a rule, e.g., aneurysm is present if the diameter of a vessel is larger than 150% compared to the average diameter of an adjacent arterial class. At Processing Block 860, the system may save volumetric calculations for the current-contour numbered r of image mask q in an open standard file format (e.g., JSON and/or Dictionary) in a database/filesystem 880. Before processing the next contour in the image mask for the current-image-file number q, in the current-arterial-class n, for the current-region i of the peripheral arterial system being processed, system is configured to increment the current-contour number r by 1 in step 862. Following this increment, at conditional block 865, the system may compare the current-contour number r with the threshold value for the total number of contours in the image mask for the current-image-file number q, in the current-arterial-class n, for the current-region i of the peripheral arterial system before proceeding further. If the current-contour number r is less than or equal to the threshold value for the total number of contours present in the image mask for the current-image-file number q, in the current-arterial-class n, for the current-region i of the peripheral arterial system, next iteration may be initiated from the connector block 850 with the incremented value of r. Otherwise, if r is greater than the threshold value for the total number of contours for an image mask, the system may terminate the loop. However, this is merely an example of how volumetric calculations may be performed for each contour in an image mask, and the results of these calculations saved, and the scope of the claimed subject matter is not limited in this respect.

Referring to FIG. 38, diagram 905 is a high-level flowchart illustrating an overview of example embodiments for a Report Generation Engine 900, utilizing a Small Language Model (SLM), to generate multiple reports for an arterial class of a region of the peripheral arterial system of a known patient. List of reports generated by the Report Generation Engine 900 may include but not limited to Volumetric Reports, Diagnostic Reports, and Vascular Arterial Surgery Planning (VASP) Reports. One or more reports may be generated by the Report Generation Engine 900 for the current-arterial-class n, in the current-region i of the peripheral arterial system, At Processing Block 910, the configured system may retrieve dictionaries with volumetric calculations data for said arterial class n in a said region i, from a plurality of dictionary databases/filesystems: 875 (for normal image masks without anomalies) and 880 (for abnormal masks with anomalies). At processing block 912, as discussed herein, the configured system may further comprise of methods that may calculate stenosis by percentage, and/or detect the cause of the stenosis (e.g., non-calcified plaque and/or calcified plaque), and/or compute the length of occlusion(s) in millimeters (mm). In some embodiments, the configured system may calculate the stenosis percentage based on the NASCET (The North American Symptomatic Carotid Endarterectomy Trial) criterion. The thickness of the tissue represented in an image slice of a CT scan may be varied to achieve an optimal balance of reduced image noise and improved diagnostic accuracy. Generally, thickness of the tissue represented in an image may range from 1 to 10 millimeters. Length of an occlusion may be computed by multiplying the number of images in the database/filesystem 880 showing occlusion with the thickness of the image slice measured in millimeters. Mathematically, the occlusion length is calculated as: Occlusion Length in millimeters=Number of Images with occlusion * Thickness of the image slice in mm. The configured system may save calculated values of detected arterial stenosis, cause of the stenosis (e.g., presence of calcified/non-calcified plaque), and/or arterial occlusion in a database, referred to as Volumetric Dictionary 914, prior to displaying the Volumetric Report as illustrated in FIG. 39 as an example. However, this is merely an example of how arterial class dictionaries may be used, how arterial stenosis and its cause may be detected, how the percentage of arterial stenosis and length of arterial occlusions may be calculated for an arterial class of a region of the peripheral arterial system, and how the results may be saved and displayed, and the scope of the claimed subject matter is not limited in this respect.

In one or more embodiments, at Processing Block 915, the configured system may input the Volumetric Dictionary 914, of an arterial class to an AI-based Small Language Model (SLM) that may have been trained for generating structured medical reports based on the volumetric calculations for an arterial class in a region of the peripheral arterial system. As discussed herein, the input Volumetric Dictionary 914 may contain calculated volumetric data, and whereas the pre-trained Small Language Model may generate a descriptive dictionary inclusive of Volumetric Report, Diagnostic Summary, and Vascular Arterial Surgery Planning (VASP) Summary, which may provide comprehensive analytics and insights, in a structured manner in a natural language form, for the clinicians and medical researchers including diagnostic radiologists, interventional radiologists, and/or vascular surgeons in detecting and reporting anomalies, e.g., occlusion, stenosis, non-calcified plaque, calcified plaque, aneurysm, etc. Such analytics, insights and recommendations may ultimately enhance the diagnostic accuracy for a patient, and facilitating timely medical interventions. Additionally, the Small Language Model may be integrated as an agentic Al, enabling autonomous decision-making that is powered by contextual learning from user feedback, and adaptive refinement of diagnostic suggestions based on historical and real-time volumetric inputs. The output of the Small Language Model for an arterial class of a region of the peripheral arterial system may be saved in a raw data format, and/or saved in a data dictionary or JSON file, and/or saved in a database/filesystem 920, referred herein as a Descriptive Dictionary. However, this is merely an example how a Small Language Model may be used to generate analytics, insights and recommendations, and the scope of the claimed subject matter is not limited in this respect.

In some embodiments, at Processing Block 930, the system may retrieve the Descriptive Dictionary data generated by the Small Language Model and saved in a Descriptive Dictionary 920. Retrieved Descriptive Dictionary data may be communicated to a processing block 935 for report generation and/or printing purposes. The reports may include a Volumetric Report, a Diagnostic Report, and/or a Vascular Arterial Surgery Planning (VASP) Report. The configured system may forward the generated Volumetric Report to a display device 940 for visualization purposes, and/or may communicate it to a printer 960. The example of a Volumetric Report as illustrated in FIG. 39, may comprise multiple arterial classes of a region along with their respective calculations, including the causes of stenosis, cumulative stenosis percentage, range of image slices with stenosis, range of image slices with occlusion, length of occlusion in millimeters and arterial diameter. The configured system may forward the generated Diagnostic Report to a display device 945 for visualization purposes, and/or may communicated it to a printer 980. An example of a Diagnostic Report is illustrated in FIG. 40. The configured system may forward the generated Vascular Arterial Surgery Planning (VASP) report to a display device 950 for visualization purposes, and/or may be communicated it to a printer 955. An example of a VASP report is illustrated in FIG. 41. However, this is merely an example of how multiple reports may be generated and how they may be displayed and/or printed, and the scope of the claimed subject matter is not limited in this respect.

In one or more embodiments, one or more computer-implemented methods may be used, as part of a clinical workflow for visualizing CTA scans in DICOM format overlaid by the results of the above disclosed methods for performing medical image analysis and diagnosis. An example of visualizing AI-generated output for the lower limb arterial system overlaid over the images in DICOM format is illustrated in FIG. 42. System may be configured for visualization purposes with a plurality of views including the example views (labeled as V1, V2, V3, and V4). One of the views (e.g., V1 in FIG. 42) may display machine-generated (Primary) CTA scans for a known patient. After converting DICOM images into 2-dimensional PNG format, the system may generate a resultant CTA scan or angiogram (in PNG format) for display with an overlay of their respective image masks. The overlay of image masks on top of the PNG images may identify arterial conditions such as blood flow, non-calcified plaque, calcified plaque, and/or aneurysm. The series of PNG images, along with their respective image masks may undergo a structured processing pipeline to generate a visual output in DICOM format for visualization using an industry-standard DICOM Viewer. One or more contour overlay techniques may merge these image masks with their corresponding converted PNG images of CTA scans, further enhancing visualization by assigning predefined colors (e.g., yellow for non-calcified plaque, red for blood flow, and/or white for calcified plaque) while detecting aneurysms based on arterial diameter analysis, particularly for the aorta. The configured system may overlay the extracted boundary coordinates in the image mask from modified region's masks database/filesystem, on the respective converted 2-dimensional (2D) PNG images, enabling precise visual differentiation of arterial features. These overlaid images may be converted into DICOM format and may be displayed along with appropriate legends in one of the views (e.g., V2 in FIG. 42) for providing enhanced diagnostic assessment. The system may stack the image masks in numerical order to generate a 2D visualization of the entire artery, which, when applied to all arterial classes, may produce a comprehensive 2D arterial system representation, such as the lower limb arterial system. This reconstructed arterial system may subsequently be converted into DICOM format and may be visualized in another view (e.g., V3 in FIG. 42) using a DICOM viewer. The dimensions of 2D resultant images are mathematically determined where Height=(Total number of images×DICOM image's slice thickness in mm) and Width=(image width in pixels×α), where α may have a range of numerical values (e.g., 1 to 1.5), depending on the slice thickness of a DICOM image. A moveable slider bar is provided to navigate through the plurality of rendered visuals. The movement of the slider button in the slider bar, in the primary image, may be synchronized to the corresponding slices in the resultant DICOM series of images. This example view (e.g., V3 in FIG. 42) may also be overlaid with a horizontal bar for the user to assist in communicating details of the area being analyzed as well as for navigating through the reconstructed 2D image with the use of a slider. The reconstructed 2D image may render a zoomed-in view for detailed visualization of rendered predictions as exhibited in example view (e.g., V4 in FIG. 42). This capability allows the user to validate the resultant images against original images to visually validate system's predictions. However, this is merely an example of how different views for the peripheral arterial system may be constructed and visualized, and the scope of the claimed subject matter is not limited in this respect.

Although the claimed subject matter has been described with a certain degree of particularity, it should be recognized that elements thereof may be altered by persons skilled in the art without departing from the spirit and/or scope of the claimed subject matter.

It is believed that diagnosing Peripheral Arterial disease using medical image analysis, risk analysis, decision making, and/or disease tracking, and/or many of its attendant advantages will be understood by the forgoing description, and it will be apparent that various changes may be made in the form, construction and/or arrangement of the components thereof without departing from the scope and/or spirit of the claimed subject matter or without sacrificing all of its material advantages, the form herein before described being merely an explanatory embodiment thereof, and/or further without providing substantial change thereto. It is the intention of the claims to encompass and/or include such changes.

It is believed that the methods of the present invention, and/or their variations, may also be applied, without departing from the scope and/or spirit of the claimed subject matter, for analyzing images and diagnosing other abnormalities including but not limited to the abnormalities in viscera, such as thoracic viscera (e.g., lungs, heart, and esophagus), and abnormalities in abdominal viscera (e.g., liver, kidney, pancreas, spleen, and intestines), as well as abnormalities in solid organs. Although the disclosed subject matter is directed to the detection and evaluation of peripheral arterial disease using CT angiography and related imaging modalities, underlying methods and techniques may be further extended and refined to support diagnostic assessments in a plurality of other territories including vascular and visceral territories.

Further, while the methods described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended embodiments. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner skilled in the art; however, the methods can also include any third-party instruction of those actions, either expressly or by implication.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments may be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

What is claimed is:

1. A computer-implemented method for diagnosing disease using medical imaging data, the method comprising:
receiving DICOM files of a plurality of anonymized patients from a CT/CAT scanner and generating PNG images data for one or more regions of the peripheral arterial system of the anonymized patients;
structuring said PNG images data and using it to train a neural network recognition model to recognize one or more regions of the peripheral arterial system;
labeling and structuring said PNG images data and using it to train a plurality of region-specific neural network classification models to categorize and classify arteries and arterial classes present in one or more regions of the peripheral arterial system;
structuring data comprising labeled PNG images and segmented image masks, and using said data to train a plurality of region-specific neural network segmentation models to segment images for one or more regions of the peripheral arterial system;
processing said PNG images data and corresponding segmented image masks to train a plurality of region-specific neural network artery labeling models to label each arterial class and/or artery and generate bounding boxes around said arterial class and/or artery for one or more regions of the peripheral arterial system;
receiving DICOM files for one or more regions of the peripheral arterial system of a known patient and generating PNG images data for the known patient;
running the neural network recognition model on the PNG images data of the known patient to recognize one or more regions of the peripheral arterial system in the PNG images data;
running a plurality of segment-specific neural network classification models using said PNG images data of the known patient to categorize arteries and arterial classes present in one or more regions of of the peripheral arterial system;
running a plurality of segment-specific neural network segmentation models using said PNG images data of the known patient to segment images for one or more regions of the peripheral arterial system;
running a plurality of segment-specific neural network artery labeling models using said PNG images data of the known patient to label each arterial class and/or artery and generate bounding boxes around said arterial class and/or artery for one or more regions of the peripheral arterial system;
integrating partial predictions made by a plurality of neural network classification, segmentation, and artery labeling models using images data of the known patient; and
running an inference engine on the integrated predictions for the known patient to diagnose disease, using a report generation engine to generate medical reports, and using a visualizion tool to view results superimposed on top of the images data for the known patient.

2. The computer-implemeneted method of claim 1, further comprising applying an algorithm that extracts axial views of DICOM images of a plurality of anonymized patients taken during the arterial phase of the scanning process, and converts said axial views into a 2-dimensional PNG image format while mapping radiodensities in Hounsfield Units to grayscale pixel values in PNG images.

3. The computer-implemeneted method of claim 1, wherein structuring PNG images data of anonymized patients and using it to train a neural network recognition model comprises:
organizing said PNG images data in a plurality of region-specific folders wherein each region-specific folder comprises PNG images for that region of the peripheral arterial system and data about the start and end points of arterial classes relative to one or more landmarks in human anatomy; and
using PNG images and data about the start and end points of arterial classes in a plurality of region-specific folders to train a neural network recognition model to recognize one or more regions of the peripheral arterial system in PNG images in addition to learning to recognize start and end points of arterial classes in each region of the peripheral arterial system.

4. The computer-implemeneted method of claim 1, wherein labeling and structuring said PNG images data and using it to train a plurality of region-specific neural network classification models to categorize and classify arteries comprises:
organizing said PNG images data and data about the start and end points of arterial classes in a plurality of region-based folders and subfolders wherein each subfolder represents the PNG images and data about the start and end points of one of the plurality of arterial classes in each region of the peripheral arterial system; and
using a plurality of region-based folders and subfolders for arterial classes in each region of the peripheral arterial system to train a plurality of region-specific neural network classification models wherein each classification model categorizes arteries and arterial classes present in said regions of the peripheral arterial system.

5. The computer-implemeneted method of claim 1, wherein structuring data comprising labeled PNG images and segmented image masks, and using said data to train a plurality of region-specific neural network segmentation models comprises:
differentiating and labeling each key feature in each PNG image based upon said feature's pixel intensity wherein a plurality of key features comprises arterial boundary, blood flow, calcified plaque, and non-calcified plaque; and
using labelled PNG images and their image masks that are stored in a plurality of region-based folders to train a plurality of region-specific neural network segmentation models to segment images and label key features.

6. The computer-implemeneted method of claim 1, wherein processing said PNG images data and corresponding segmented image masks to train a plurality of region-specific neural network artery labeling models comprises:
labeling said PNG images and generating image masks for each region of the peripheral arterial system;
applying a Bitwise AND Operation on each PNG image and its image mask to generate a resultant arterial image; and
structuring and organizing labels data and said resultant arterial images in region-specific folders, and using label data and resultant arterial images to train a plurality of region-specific neural network artery labeling models to detect and label arterial objects of interest in each image and draw boundaries around said arterial objects.

7. The computer-implemented method of claim 1, wherein each neural network model is a residual neural network model which is configured to skip one or more intermediate nodes in a layered convolutional neural network.

8. The computer-implemented method of claim 1, wherein each artery labeling model is configured as a Faster R-CNN with a Feature Pyramid Network (FPN), which comprises a backbone residual neural network, a region (of interest) proposal network (RPN), a region of interest (ROI) pooling layer, an object detection and classification layer, and a bounding box regression head.

9. The computer-implemented method of claim 1, further comprising applying an algorithm that extracts axial views of DICOM images of a known patient taken during the arterial phase of the scanning process, converts said axial views into a 2-dimensional PNG image format while mapping radiodensities in Hounsfield Units to grayscale pixel values in PNG images, and uses a pre-trained neural network recognition model on each PNG image which predicts the region of the peripheral arterial system for each PNG image, and stores each PNG image in a region-specific folder.

10. The computer-implemented method of claim 1, further comprising applying an algorithm that iterates over each region of the peripheral arterial system of the known patient, uses a pre-trained region-specific neural network classification model to predict and categorize each arterial class that is present in each PNG image, saves the results predicted by said classification model, uses a noise filtering method to remove inconsistencies in the arterial classes data predicted by said classification model, uses a pre-trained region-specific neural network segmentation model to create an image mask by segmenting each PNG image, saves the image mask alongwith the labels for each key feature for diagnosing disease, and uses a noise filtering method to remove inconsistencies from the image masks predicted by said segmentation model.

11. The computer-implemented method of claim 10, further comprising applying an algorithm that iterates over each image and its image mask in the current region of the peripheral arterial system of the known patient, uses a Bitwise AND Operation on each PNG image and its image mask to generate a resultant arterial image, uses a pre-trained region-specific neural network artery labeling model on the resultant arterial image to predict and label each artery and generate a bounding box around the area of the predicted artery in the resultant arterial image, and saves the predicted results.

12. The computer-implemented method of claim 10, further comprising applying an algorithm that iterates over each PNG image in the current region of the peripheral arterial system of the known patient, retrieves its DICOM tags data, retrieves predictions made by a pre-trained neural network recognition model, retrieves predictions made by a plurality of region-specific neural network models (comprising classification, segmentation, and arterial labeling models), applies Anekanta algorithm with rules configured to integrate predictions data, and saves integrated predictions data.

13. The computer-implemented method of claim 10, further comprising applying an inference engine algorithm that iterates over each arterial class in the current region of the peripheral arterial system to detect disease of the known patient, which in turn iterates over each image within each arterial class being processed, retrieves integrated predictions data generated by the Anekanta algorithm, retrieves modified image mask data, detects one or more anomalies in the modified image mask data wherein each anomaly has a pixel intensity that is different from the pixel intensity for blood, differentiates each area with a uniform pixel intensity with a countour around it, assigns a semantic value (list of semantic values comprises non-calcified plaque, calcified plaque, and blood flow) to each area within each contour, performs volumetric calculations for each anomaly detected, and saves the results in a database/filesystem.

14. The computer-implemented method of claim 10, further comprising applying a report generation engine algorithm that iterates over each arterial class in the current region of the peripheral arterial system to report disease for the known patient, retrieves results generated by the inference engine, calculates stenosis if present by percentage, detects the cause of the stenosis if present, computes the length of occlusion if present, and uses a small language model to prepare a plurality of reports (list of reports comprises a Volumetric Report, Diagnostic Summary, and Vascular Arterial Surgery Planning (VASP) Summary).

15. The computer-implemented method of claim 10, further comprising applying a visualization algorithm to display a plurality of views of peripheral arterial system of the known patient in a DICOM viewer wherein one of the views is configured to display primary CTA scan images of the known patient, another view is configured to display images overlaid by a plurality of area to highlight arterial conditions in different colors, another view is configured to display a 2D visualization of the entire peripheral arterial system with a method to help a clinician move a horizontal bar to select a position in an area of interest for visualization purposes along with another view that displays a zoomed-in view of the area of interest that has been selected by the clinician.

16. A system for diagnosing disease using medical imaging data, comprising:
one or more processor nodes wherein each processor node is coupled with at least one network interface, a filesystem/database, and memory having program instructions stored therein that are executable by the processor nodes to cause the system to execute:
means for receiving DICOM files of a plurality of anonymized patients from a CT/CAT scanner and generating PNG images data for one or more regions of the peripheral arterial system of the anonymized patients;
means for structuring said PNG images data and using it to train a neural network recognition model to recognize one or more regions of the peripheral arterial system;
means for labeling and structuring said PNG images data and using it to train a plurality of region-specific neural network classification models to categorize and classify arteries and arterial classes present in one or more regions of of the peripheral arterial system;
means for structuring data comprising labeled PNG images and segmented image masks, and using said data to train a plurality of region-specific neural network segmentation models to segment images for one or more regions of the peripheral arterial system;
means for processing said PNG images data and corresponding segmented image masks to train a plurality of region-specific neural network artery labeling models to label each arterial class and/or artery and generate bounding boxes around said arterial class and/or artery for one or more regions of the peripheral arterial system;
means for receiving DICOM files for one or more regions of the peripheral arterial system of a known patient and generating PNG images data for the known patient;
means for running the neural network recognition model on the PNG images data of the known patient to recognize one or more regions of the peripheral arterial system in the PNG images data;
means for running a plurality of segment-specific neural network classification models using said PNG images data of the known patient to categorize arteries and arterial classes present in one or more regions of the peripheral arterial system;
means for running a plurality of segment-specific neural network segmentation models using said PNG images data of the known patient to segment images for one or more regions of the peripheral arterial system;
means for running a plurality of segment-specific neural network artery labeling models using said PNG images data of the known patient to label each arterial class and/or artery and generate bounding boxes around said arterial class and/or artery for one or more regions of the peripheral arterial system;
means for integrating partial predictions made by a plurality of neural network classification, segmentation, and artery labeling models using images data of the known patient; and
means for running an inference engine on the integrated predictions for the known patient to diagnose disease, using a report generation engine to generate medical reports, and using a visualizion tool to view results superimposed on top of the images data for the known patient.

17. A non-transitory computer readable medium storing computer executable instructions for diagnosing disease using medical imaging data, the computer executable instructions when executed by one or more processors perform operations comprising:
receiving DICOM files of a plurality of anonymized patients from a CT/CAT scanner and generating PNG images data for one or more regions of the peripheral arterial system of the anonymized patients;
structuring said PNG images data and using it to train a neural network recognition model to recognize one or more regions of the peripheral arterial system;
labeling and structuring said PNG images data and using it to train a plurality of region-specific neural network classification models to categorize and classify arteries and arterial classes present in one or more regions of of the peripheral arterial system;
structuring data comprising labeled PNG images and segmented image masks, and using said data to train a plurality of region-specific neural network segmentation models to segment images for one or more regions of the peripheral arterial system;
processing said PNG images data and corresponding segmented image masks to train a plurality of region-specific neural network artery labeling models to label each arterial class and/or artery and generate bounding boxes around said arterial class and/or artery for one or more regions of the peripheral arterial system;
receiving DICOM files for one or more regions of the peripheral arterial system of a known patient and generating PNG images data for the known patient;
running the neural network recognition model on the PNG images data of the known patient to recognize one or more regions of the peripheral arterial system in the PNG images data;
running a plurality of segment-specific neural network classification models using said PNG images data of the known patient to categorize arteries and arterial classes present in one or more regions of of the peripheral arterial system;
running a plurality of segment-specific neural network segmentation models using said PNG images data of the known patient to segment images for one or more regions of the peripheral arterial system;
running a plurality of segment-specific neural network artery labeling models using said PNG images data of the known patient to label each arterial class and/or artery and generate bounding boxes around said arterial class and/or artery for one or more regions of the peripheral arterial system;
integrating partial predictions made by a plurality of neural network classification, segmentation, and artery labeling models using images data of the known patient; and
running an inference engine on the integrated predictions for the known patient to diagnose disease, using a report generation engine to generate medical reports, and using a visualizion tool to view results superimposed on top of the images data for the known patient.
